(19) United States
(12) Patent Application Publication
(10) Pub. No.: US 2024/0309321 A1
(43) Pub. Date: Sep. 19, 2024

(54) DEVELOPMENT OF AN ACCELERATED CELLULAR MODEL FOR EARLY CHANGES IN ALZHEIMER’S DISEASE

(71) Applicant: University of Maryland, College Park, College Park, MD (US)

(72) Inventors: Kan Cao, Bethesda, MD (US); Huijing Xue, Rockville, MD (US); Wolfgang Losert, Bethesda, MD (US)

(73) Assignee: University of Maryland, College Park, College Park, MD (US)

(21) Appl. No.: 18/593,295

(22) Filed: Mar. 1, 2024

Related U.S. Application Data
(60) Provisional application No. 63/488,475, filed on Mar. 3, 2023.

(51) Int. Cl. C12N 5/0793

(52) U.S. Cl. CPC C12N 5/0619; C12N 2506/08

(57) ABSTRACT

The present disclosure provides an accelerated AD model system for use in the diagnosis and prognosis of Alzheimer's disease (AD). The AD accelerated model system may also be used in screening assays for identifying therapeutic agents that correct, or alleviate, one or more of the cell culture phenotypes associated with the development of AD. Such phenotypes include, for example, increased tau phosphorylation, amyloid plaque accumulation, elevated levels of Aβ42 to Aβ40 ratio, increased cell death and cell cycle re-entry. The methods and compositions provided herein are based on the discovery that exogenously expressed progerin in neural progenitor cells leads to a robust and accelerated AD phenotype in said cells as they differentiate and that the modified and differentiated cells surprisingly produce factors which generate an aged environment (“aging factors”) for all the cells in their vicinity.

This application claims benefit and priority to U.S. Provisional Application No. 63/488,475 filed on Mar. 3, 2023, which is incorporated herein by reference in its entirety.

### REFERENCE TO ELECTRONIC SEQUENCE LISTING

This application contains a Sequence Listing which has been submitted electronically in .XML format and is hereby incorporated by reference in its entirety. Said .XML copy, created on May 17, 2024, is named “1475-110 US.xml” and is 34,228 bytes in size. The sequence listing contained in this .XML file is part of the specification and is hereby incorporated by reference herein in its entirety.

### FIELD OF THE DISCLOSURE

The present disclosure provides an accelerated AD model system for use in the diagnosis and prognosis of Alzheimer's disease (AD). The AD accelerated model system may also be used in screening assays for identifying therapeutic agents that correct, or alleviate, one or more of the cell culture phenotypes associated with the development of AD. Such phenotypes include, for example, increased tau phosphorylation, amyloid plaque accumulation, elevated levels of Aβ42 to Aβ40 ratio, increased cell death and cell cycle re-entry. The methods and compositions provided herein are based on the discovery that exogenously expressed progerin in neural progenitor cells leads to a robust and accelerated AD phenotype in said cells as they differentiate and that the modified and differentiated cells surprisingly produce factors which generate an aged environment (“aging factors”) for all the cells in their vicinity.

### BACKGROUND

AD is a neurodegenerative disease, and one of the most common causes of dementia (Erkkinen et al., Cold Spring Harb. Perspect. Biol. 10, a033118 (2018)). It can be subtyped into two categories. One is familial AD (FAD), which usually begins before age 65, and the other is sporadic AD (SAD), which usually begins after age 65 (Bekris et al., J. Geriatr. Psychiatry Neurol. 23, 213-227 (2010)). Two hallmarks of AD are senile plaques made up of β-amyloid (AB), and neurofibrillary tangles (NFTs) made up of tau protein (Serrano-Pozo, et al. Cold Spring Harb. Perspect. Med. 1, a006189-a006189 (2011)). Both genetic and environmental risk factors can contribute to the formation of plaques and fibrillary tangles, with aging being one of the greatest risk factors (Gauthier et al. Alzheimer's Dis. Int. 25, 50 (2022). However, the specific mechanism underlying how these protein aggregations form remains unclear.

Currently, most of the AD drug candidates (>99.6% since 2002) in clinical trials fail to demonstrate sufficient clinical efficacy (Cummings, J. L. et al. Alzheimers. Res. Ther. 6, 37 (2014)). A significant challenge in Alzheimer's disease research is the absence of standardized and effective models. There are three main types of experimental models available: animal models, human post-mortem tissues, and cell culture models. Animal models are typically time-consuming and expensive. The majority of AD animal models are transgenic mice, with over 200 models developed to date (according to Alzforum). However, most clinical trials have failed, despite the efficacy of the compounds used to slow the disease in mouse models (Cummings et al. Alzheimer's Res. Ther. 6, 1-7 (2014); Banik, A. et al. J. Alzheimer's Dis. 47, 815-843 (2015)). The reasons for the lack of translational success in mouse models are complicated. Due to the sequence differences between mouse Aβ and human Aβ (Xu, G. et al. Acta Neuropathol. Commun. 3, 72 (2015)), most mouse models utilized one or multiple FAD mutations to introduce transgenic human Aβ. Although these FAD models can generate some AD features, none of them can recapitulate the complete disease profile. Most of them do not show either amyloid plaques or tau tangles (Drummond, E. & Wisniewski, T. Acta Neuropathol. 133, 155-175 (2017)). Mouse and human tau share only 88% sequence homology and endogenous mouse tau could inhibit the aggregation of human tau (Duff, K. et al. Neurobiol. Dis. 7, 87-98 (2000); Andorfer, C. et al. J. Neurochem. 86, 582-590 (2003)). This could explain the lack of tau tangles in these transgenic mice. More recently, knock-in mice were developed, which were considered more physiologically relevant (Xia, D. et al. Mol. Neurodegener. 17, 1-29 (2022)). However, the pathological phenotypes and disease progression are still inconsistent, and if these knock-in mouse models are representative still needs to be validated. Because of the lack of high-quality human post-mortem tissues, cell culture models have become a popular choice. Most AD cell culture models are derived from human induced pluripotent stem cells (iPSCs) (Penney, J., Ralvenius, W. T. & Tsai, Mol. Psychiatry 25, 148-167 (2020)). However, iPSC-derived neurons can be rejuvenated after reprogramming (Lapasset, L. et al. Genes Dev. 25, 2248-2253 (2011)). This poses a concern since AD is an age-related disease, which could explain why many current iPSC-derived models are time-consuming and fail to recapitulate the amyloid plaque and tau tangle simultaneously. The appearance of AD characteristic features in primary cell culture often requires several months (Shi, Y. et al. Sci. Transl. Med. 4, 124ra29 (2012); Kim, Y. H. et al. Nat. Protoc. 10, 985-1006 (2015); Jorfi, M., D'Avanzo, C., Tanzi, R. E., Kim, D. Y. & Irimia, D. Human Neurospheroid Arrays for In Vitro Studies of Alzheimer's Disease. Sci. Reports 2018 81 8, 1-13 (2018)), and the time for detecting these phenotypes can be unpredictable, making data reproducibility a challenge. Accordingly, development of an efficient and accelerated AD cellular model for diagnosis and drug screening is urgently needed.

### SUMMARY

The present disclosure provides methods and compositions for diagnosing or prognosing an age-related neurodegenerative disorder such as AD in a subject. Said methods and compositions can also be used in choosing a prescribed therapeutic regimen or predicting the benefit from a given therapy in a subject carrying potential risks of developing AD or having AD. The present disclosure further provides methods and compositions for identifying therapeutic agents useful for treatment of AD in a subject.

As disclosed herein, it has been discovered that when progerin is exogenously, e.g., recombinantly, expressed in neural progenitor cells said cells produce a robust AD phenotype within three to four weeks as they differentiate towards a neuronal phenotype. Such an AD phenotype includes increased tau phosphorylation, amyloid plaque accumulation, elevated levels of Aβ42 to Aβ40 ratio, increased cell death and cell cycle re-entry. Accordingly, the present disclosure provides an accelerated Alzheimer's Disease (AD) model system comprising cultured neuronal progenitor cells, or iPSCs, that have been (i) engineered to express progerin (ii) and subjected to cell culture conditions leading to neuronal differentiation and wherein said cultured cells develop one or more AD associated phenotypes.

Said model system can be utilized for use in diagnostic and drug screening assays. For example, and as disclosed in detail below, the accelerated AD model system may be used in the diagnosing or prognosing of AD in a subject wherein the iPSCs are derived from said subject and wherein detection of an increase in the level of AD associated phenotypes relative to a control biological sample indicates that the subject has AD, or is at risk of developing AD. Said model system may also be used in identifying a therapeutic agent useful for treating AD wherein said therapeutic agent corrects an AD associated phenotype comprising the additional step of contacting said cultured neuronal progenitor cells, or iPSCs, with a test therapeutic agent and identifying a therapeutic agent as correcting the AD associated phenotype if a decrease in the level of AD associated phenotypes is detected relative to a control sample.

In one embodiment, neuronal progenitor cells engineered to express progerin are provided. Additionally, for individual-specific diagnostic and drug screening, iPS-derived cells generated from a test subject and engineered to express progerin, are provided. It has also been observed that the culture medium derived from said progerin expressing cells, derived from neuronal progenitor cells or iPS cells, surprisingly secreted aging factors which generate an aged environment for all the cells in their vicinity. Accordingly, cell culture supernatants derived from said progerin expressing cells are provided that induce, in contacted cells, phenotypes associated with development of AD.

In an embodiment, the present disclosure provides methods for deriving an accelerated AD model system comprising (i) culturing neuronal progenitor cells wherein said progenitor cells have been recombinantly engineered to express progerin and (ii) subjecting the cell culture to conditions leading to neuronal differentiation. In one aspect the accelerated AD model system is successfully derived in under 6 weeks, preferably between 3-4 weeks. In another aspect the cells are cultured in 2D/3D culture. In yet another embodiment, the cells are engineered to express familial AD mutations.

In another embodiment, the present disclosure provides methods for deriving an accelerated AD model system comprising (i) culturing iPSCs derived from a subject wherein said iPSCs have been recombinantly engineered to express progerin and (ii) subjecting the cell culture to conditions leading to neuronal differentiation. In one aspect the accelerated AD model system is successfully derived in under 6 weeks, more preferably between 3-4 weeks. In another aspect the cells are cultured in 2D/3D culture. In yet another embodiment, the cells are engineered to express familial AD mutations.

In yet another embodiment, the present disclosure provides methods for deriving an accelerated AD model system comprising (i) culturing neuronal progenitor cells or iPSCs derived from a subject; (ii) subjecting the cell culture to conditions leading to neuronal differentiation and (iii) contacting the cell culture to supernatant culture medium derived from cells exogenously expressing progerin. In one aspect the accelerated AD model system is successfully derived in under 6 weeks, preferably between 3-4 weeks. In another aspect the cells are cultured in 2D/3D culture. In yet another embodiment, the cells are engineered to express familial AD mutations.

The present disclosure provides methods for diagnosing or prognosing AD in a subject comprising: (i) obtaining iPSCs derived from a subject wherein said cells have been recombinantly engineered to express progerin and subjected to cell culture conditions leading to neuronal differentiation; (ii) detecting one or more AD associated phenotypes in said cell culture; and (iii) determining that the subject has AD, or is at risk of developing AD in the future, by detecting an increase in the level of AD associated phenotypes relative to a control biological sample.

In another embodiment, methods for diagnosing or prognosing AD in a subject are provided comprising the steps of: (i) obtaining iPSC derived from the subject wherein said cells have been subjected to culture conditions leading to neuronal differentiation; (ii) contacting said cells with cell culture medium that has been derived from neuronal progenitor cells that exogenously express progerin and wherein said cell culture medium comprises secreted aging factors; (ii) detecting one or more AD associated phenotypes in said cell culture; and (iii) determining that the subject has AD, or is at risk of developing AD, by detecting an increase in the level of AD associated phenotypes relative to a control biological sample.

The present disclosure further provides methods for identifying a therapeutic agent useful for treating AD wherein said therapeutic agent corrects a phenotype associated with development of AD comprising (i) contacting a first population of cells derived from neural progenitor cells expressing exogenous progerin, with a candidate therapeutic agent; (ii) contacting a second population of cells derived from neural progenitive cells expressing exogenous progerin with a control agent; (iii) assaying the two populations and (iv) identifying a therapeutic agent as correcting the AD associated phenotype if the first population is closer to a normal phenotype following treatment than the second population.

Using iPS cells derived from a specific subject, a method is provided to identify or determine the likelihood that a particular therapeutic agent will have sufficient efficacy in that particular subject, and if so, an appropriate dose range for that subject. Accordingly, the present disclosure also provides methods for identifying a therapeutic agent that corrects a phenotype associated with development of AD in a specific subject comprising (i) contacting a first population of iPS cells derived from said subject and expressing exogenous progerin, with a candidate therapeutic agent; (ii) contacting a second population of iPS cells derived from said subject and expressing exogenous progerin with a control agent; (iii) assaying the two populations and (iv) identifying candidate agents as correcting the AD associated phenotype if the first population is closer to a normal phenotype following treatment than the second population.

The present disclosure also provides methods for identifying a therapeutic agent that corrects a phenotype associated with development of AD in a specific subject comprising (i) contacting a first population of iPS cells derived from said subject with a candidate therapeutic agent in the presence of cell culture medium that has been derived from neuronal progenitor cells that exogenously express progerin and wherein said cell culture medium comprises secreted aging factors; (ii) contacting a second population of iPS cells derived from said subject with a control agent in the presence of cell culture medium that has been derived from neuronal progenitor cells that exogenously express progerin and wherein said cell culture medium comprises secreted aging factors; (iii) assaying the two populations and (iv) identifying candidate agents as correcting the AD associated phenotype if the first population is closer to a normal phenotype following treatment than the second population.

In an embodiment, the neural progenitive cells, or iPSC cells, for use in the disclosed diagnostic and screening methods comprise a familial AD mutation. In one aspect, the AD associated phenotype for both the diagnostic and drug screening assays includes, but is not limited to, increased tau phosphorylation, amyloid plaque accumulation, elevated levels of Aβ42 to Aβ40 ratio, increased cell death and cell cycle re-entry.

Additionally, for each of the diagnostic/prognostic and drug screening assays disclosed herein said assays may, for example, be performed advantageously within the 3-4 week timeframe it takes for development of the accelerated AD model system. Alternatively, said assays may be performed any time after development of AD associated phenotypes.

The present disclosure also provides kits for diagnosing or prognosing AD in a subject, identifying a subject at risk of development of AD, or identifying a therapeutic regimen or predicting benefit from therapy in a subject having AD, the kit comprising one or more containers for collecting and or holding a biological sample, and an instruction for its use, as well as reagents for detecting AD associated phenotypes.

### BRIEF DESCRIPTION OF THE DRAWINGS

FIG. 1A-E. The expression of nuclear lamins mRNA and protein during the differentiation. (FIG. 1A) The quantification of mRNA relative expression of lamin A, lamin C and lamin B1 at different time points (day 0, day 7, day 14). Both lamin A and lamin B were significantly decreased while there was no significant change in lamin C mRNA. (FIG. 1B) Immunofluorescence staining of neuronal markers and lamin A/C at day 14. β-tubulin III and MAP2 are neuronal markers and GFAP is astrocytes markers. Lamin A/C staining is also positive in ReN cells after two-week differentiation. (Scale bar: 20 μm) (FIG. 1C-D) Western blot results of lamin A, lamin C and lamin B1 protein levels at different time points (day0, day7, day14). Both lamin A and lamin C were downregulated while lamin B1 was relatively stable. (FIG. 1E) Summary of the mRNA and protein expression pattern of nuclear lamins during NPC differentiation. All the results were generated from more than three biological replicates. Asterisks indicate statistical difference as follows: ns, not significant; *p<0.05; **p<0.01; ***p<0.001; ****p<0.0001.

FIG. 2A-D. The effects of the overexpression of lamin A and progerin in neural cells. (FIG. 2A) ROS level after two-week lamin A and progerin overexpression. ROS level was significantly increased with progerin expression. (FIG. 2B) The quantification of cell death assay. The percentage of early cell death was significantly increased with progerin expression after two-week differentiation. (FIG. 2C) The quantification of cell cycle assay. The percentage of S-phase cells was increased after two-week lamin A- or progerin-transduction. (FIG. 2D) Immunofluorescence staining of BrdU in differentiated cells. Blue indicated DAPI signal, green indicated GFP-tagged lamin A or progerin, pink indicated BrdU signal. The percentage of BrdU positive cells over DAPI positive cells was increased after lamin A- or progerin-transduction. All the results were generated from three biological replicates. *p<0.05. (Scale bar: 20 μm)

FIG. 3A-E. (FIG. 3A) Experimental timeline. ReN cells were transduced with the lentivirus containing FAD mutation constructs before the differentiation. Cells were seeded on Matrigel-coated plates for 2D culture while cell suspension was mixed with Matrigel in 3D culture. After two-week differentiation, lamin A or progerin expression were transduced in the cells and followed by downstream analyses. Samples were as listed. (FIG. 3B-C) Protein level of total tau and phosphorylated tau after 4 weeks. The total tau level was not significantly changed. Phosphorylated tau was increased after lamin A expression, and progerin expression further upregulated tau phosphorylation significantly. Results were generated from four biological replicates. n.s., not significant; *p<0.05. (FIG. 3D) Aβ42/Aβ40 ratio after 3-week differentiation. Within the mAP group, Aβ42/Aβ40 ratio was slightly increased after lamin A expression and significantly increased after progerin expression. Results were generated from three biological replicates. *p<0.05. (FIG. 3E) Aβ aggregation staining with Amylo-glo after 4-week differentiation. Blue indicated the Amylo-glo staining, green indicated the GFP-tagged lamin A or progerin, red indicated mcherry or mcherry-tagged APP and PSEN1. Yellow arrows indicated the Aβ fibrils. (Scale bar: 20 μm)

FIG. 4A-D. Increased cell cycle re-entry and cell death after FAD mutations and progerin co-transduction after 4 weeks. (FIG. 4A) The quantification of cell cycle assay. Within both mcherry control group and mAP group, S phase cells were increased after lamin A- and progerin-transduction. Comparing mAP cells to mcherry control cells, S phase percentage was increased as well. (FIG. 4B) The quantification of BrdU positive cells. An increased percentage of BrdU positive cells over DAPI positive cells after lamin A- and progerin-transduction was observed in both mcherry control group and mAP group. (FIG. 4C) The quantification of cell death flow cytometry. Cell death was increased comparing mAP group to mcherry control group. Within each group, progerin expression significantly induced more cell death. (FIG. 4D) The quantification of mRNA relative expression of YAP. YAP mRNA was downregulated in mAP ReN cells and progerin expression could further decrease YAP expression. All the results were generated from three biological replicates. n.s., not significant; *p<0.05; **p<0.01; ***p<0.001; ****p<0.0001.

FIG. 5A-B. Model of accelerated aging with progerin intervention. (FIG. 5A) We hypothesized that progerin could provide an aging environment for neurodegeneration. Cells containing only FAD mutations are not typically subject to aging in most circumstances. The connection between nucleoskeleton and cytoskeleton is integrated, and cell mechanic is well maintained. After progerin expression, the balance of the nucleoskeleton is disrupted and the cell environment could be stiffer, which makes cells vulnerable and results in more protein aggregation, cell cycle re-entry and cell death. (FIG. 5B) Comparison of schematic timeline between acAD model and a 3D model from Kim et al., 2015. Neural progenitor cells are differentiated from Week 0 in both protocols. Amyloid plaques and phosphorylated tau, two important AD hallmarks, were observed in the acAD model after 4-week differentiation, while it takes much longer in a well-characterized 3D AD model.

FIG. 6A-C. The process of ReN cell differentiation. (FIG. 6A) ReN cells could grow neurites in two weeks. (Scale bar: 100 μm). (FIG. 6B) Differentiated cells were positive with neuronal markers, MAP2 and astrocyte markers, GFAP. (Scale bar: 20 μm). (FIG. 6C) Differentiated cells could generate Ca2+ transients in the Tyrode's solution supplemented with Na+.

FIG. 7A-E. Lamin A- and progerin-transduction in ReN cells. (7A) Diagrams of lentiviral constructs. GFP-LA virus contains lamin A cDNA and GFP-Pg virus contains progerin cDNA. Both vectors contain EGFP signal. (7B-C) Quantification of transduction efficiency. The transduction was successful, and the efficiency was 71.1% for GFP-LA virus and 64.8% for GFP-Pg virus after 2-day transduction. (7D-E) Western blot results of exogenous protein level and GFP signals during the differentiation. Exogenous progerin protein were decreased during the differentiation.

FIG. 8A-B Representative flow cytometry plots. (FIG. 8A) Annexin V/PI flow cytometry plots. (FIG. 8B) Cell cycle flow cytometry analysis with Dean-Jett-Fox model.

FIG. 9. Nuclear morphology changes after progerin transduction. Abnormal nuclear morphology was only observed after progerin expression but not after lamin A expression. White arrows point to the nucleus with severe nuclear morphology changes. (Scale bar: 20 μm).

FIG. 10A-E. Combination of FAD mutations and progerin-transduction in ReN cells. (FIG. 10A) Diagrams of lentiviral constructs. A plasmid containing APP with both the K670N/M671L (Swedish) and V717I (London) mutations (APPSL) and PSEN1 with the 49 mutation (PSEN1(49)) was a gift from Dr. Kim's lab. (FIG. 10B) Quantification of mRNA level after FAD transduction. The transcription level of both APP and PSEN1 was upregulated after the transduction. (FIG. 10C) Western blot of exogenous lamin A and progerin. After 2-day transduction, both lamin A and progerin expression were abundant. After 2 weeks, lamin A level was still abundant, while progerin level became very weak. (FIG. 10D) Protein concentration of Aβ40 and 42 in the culture medium after 3-week culture. (FIG. 10E) All and GFP-LA/Pg immunofouresence.

FIG. 11. Aβ oligomer staining after 3-week differentiation. Pink indicated Aβ oligomer staining, red indicated mcherry signal, green indicated the GFP-tagged lamin A or progerin, blue indicated DAPI signal (Scale bar: 20 μm).

FIG. 12A-C. The mRNA expression of cell-cycle-related regulators after 4 weeks. (FIG. 12A). Quantification of p16 mRNA expression. Within mcherry and mAP groups, p16 mRNA level was significantly increased after lamin A expression. Same trend was observed after progerin expression. Results were generated from four biological replicates. n.s., not significant; *p<0.05; **p<0.01; ***p<0.001; ****p<0.0001. (FIG. 12B). Quantification of cdk4 mRNA expression. Within mcherry and mAP groups, cdk4 mRNA level was significantly increased after lamin A expression. Same trend was observed after progerin expression. Results were generated from three biological replicates. n.s., not significant; *p<0.05; **p<0.01; ***p<0.001; ****p<0.0001. (FIG. 12C). Quantification of cdk6 mRNA expression. mRNA level of cdk6 was higher in the cells with FAD mutants alone than the cells with mcherry control alone. Within mcherry and mAP groups, cdk6 mRNA level was significantly increased after lamin A expression. Same trend was observed after progerin expression. Results were generated from three biological replicates. n.s., not significant; *p<0.05; **p<0.01; ***p<0.001; ****p<0.0001.

FIG. 13A-C. DNA damage and telomere length after progerin addition. (FIG. 13A-B). Western blot of γH2AX. γ-H2AX was upregulated after ectopic lamin A expression in both mcherry control group and mAP group after 4 weeks. Same trend was observed after progerin expression. Results were generated from three biological replicates. n.s., not significant; *p<0.05; **p<0.01; ***p<0.001; ****p<0.0001. (FIG. 13C). Quantification of telomere length with qPCR after 4 weeks. Telomere length did not show significant change with FAD intervention or progerin intervention. Results were generated from three biological replicates. n.s., not significant.

FIG. 14A-C. Condition Medium experiment suggests the presence of soluble aging factors in progerin-expressing cell media. (FIG. 14A). Western blotting analysis of Tau Thr231 phosphorylation (p-Tau) in AD cells, AD cells treated with lamin A (LA)-conditioned media, and PG-conditioned Media. (FIG. 14B). Ratios of p-Tau to total Tau in each sample. (FIG. 14C). Quantitative RT-PCR of p16.

FIG. 15. Assay primer list (SEQ ID NOS:3-24).

### DETAILED DESCRIPTION

The following is a detailed description of the novel compositions and methods for soft tissue augmentation. Unless otherwise defined, all technical and scientific terms used herein have the same meaning as commonly understood by one of ordinary skill in the art to which this disclosure belongs. The terminology used in the present disclosure is for describing particular embodiments only and is not intended to be limiting to the disclosure. All publications mentioned herein are incorporated herein by reference to disclose and describe the methods and/or materials in connection with which the publications are cited.

The following disclosure provides methods and composition for use in diagnosing and drug discovery as they relate to Alzheimer's disease (AD), however, it is understood that said disclosure may be applied equally as well to other neurodegenerative disorders, including but not limited to vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis andtraumatic brain injury (TBI).

As disclosed herein, it has been observed that when progerin is exogenously, e.g., recombinantly, expressed in neural progenitor cells said cells produce a robust AD phenotype within three to four weeks as they differentiate towards a neuronal phenotype. Such an AD phenotype includes increased tau phosphorylation, amyloid plaque accumulation, elevated levels of Aβ42 to A β42 ratio, increased cell death and cell cycle re-entry. Accordingly, the present disclosure provides an accelerated model for AD development for use in diagnostic and drug screening assays.

In an embodiment, the present disclosure provides methods for deriving an accelerated AD model system comprising (i) culturing neuronal progenitor cells wherein said progenitor cells have been recombinantly engineered to express progerin and (ii) subjecting the cell culture to conditions leading to neuronal differentiation. In one aspect the accelerated AD model system is successfully derived in under 6 weeks, preferably between 3-4 weeks. In another aspect the cells are cultured in 2D/3D culture. In yet another embodiment, the cells are engineered to express familial AD mutations.

In another embodiment, the present disclosure provides methods for deriving an accelerated AD model system comprising (i) culturing iPSCs derived from a subject wherein said iPSCs have been recombinantly engineered to express progerin and (ii) subjecting the cell culture to conditions leading to neuronal differentiation. In one aspect the accelerated AD model system is successfully derived in under 6 weeks, preferably between 3-4 weeks. In another aspect the cells are cultured in 2D/3D culture. In yet another embodiment, the cells are engineered to express familial AD mutations.

In yet another embodiment, the present disclosure provides methods for deriving an accelerated AD model system comprising (i) culturing neuronal progenitor cells or iPSCs derived from a subject; (ii) subjecting the cell culture to conditions leading to neuronal differentiation and (iii) contacting the cell culture to supernatant culture medium derived from cells exogenously expressing progerin. In one aspect the accelerated AD model system is successfully derived in under 6 weeks, preferably between 3-4 weeks. In another aspect the cells are cultured in 2D/3D culture. In yet another embodiment, the cells are engineered to express familial AD mutations. In one aspect, neuronal progenitor cells are provided that have been genetically engineered to express progerin. Said neuronal progenitor cells include, but are not limited to, ReNcell VM immortalized human neural progenitor cells (hNPC). Additional neuronal progenitor cells, known to those of skill in the art, may be used equally as well in the methods provided herein. In another embodiment, the neuronal progenitor cells are further recombinantly engineered to express familial AD mutations in addition to progerin. Such familial AD mutations include, but are not limited to, mutations in genes Presenilin 1 (PSEN1), Presenilin 2 (PSEN2), and Amyloid precursor protein (APP). In another aspect, for individual specific diagnostic and drug screening, iPS-derived cells generated from a test subject and engineered to express progerin, are also provided.

Differentiated cells can be reprogrammed to an embryonic-like state. (See, for example, Takahashi and Yamanaka Cell 2006 126:663-676). Such induction of pluripotent stem cells from adult fibroblasts can be accomplished, for example, by introducing four factors, Oct3/4, Sox2, c-Myc, and Klf4, under ES cell culture conditions. These cells, designated iPS cells, exhibit the morphology and growth properties of ES cells and express ES cell marker genes. In some cases, iPS cell lines are generated specifically from individuals in need of diagnosis or prognosis of AD or for use in personalized drug screening.

In one aspect, production of the accelerated AD model proceeds in a time frame of under six weeks or about 3-5 weeks. For culturing of each of the different cells, e.g . . . , neuronal progenitor cells or iPS cells, in preparation for use in the diagnostic or drug screening assays disclosed herein, a non-limiting culturing time table may be as follows: (i) seeding the hNPCs or iPS cells on Day-3; (ii) placement in 2D/3D culture on Day 0; (iii) start of differentiation on Day 1; recombinant introduction of progerin into the cultured cells Week 2; and AD phenotype detection Weeks 3-4. It is understood that such a timetable is provided only as an example. and the timing of the steps may be altered resulting, nevertheless, in the derivation of cells exhibiting AD culture phenotypes.

For both neuronal progenitor cells and iPSC, a step is included where the cells are cultured under conditions leading to neuronal differentiation. Such differentiation can be accomplished using specific differentiation media or media supplemented with differentiation factors know to those skilled in the art for neuronal differentiation. For example, BrainPhys Neuronal Medium (STEMCELL) lacking bFGF and EGF may be used to differentiate cells.

It has also been observed that the culture medium derived from said progerin engineered cells, e.g., derived from neuronal progenitor cells or iPSC cells, surprisingly produce secreted aging factors which generate an aged environment for all the cells in their vicinity. Accordingly, provided are culture supernatants derived from said progerin engineered cells that show the culture phenotypes associated with development of AD. Such supernatants may be advantageously utilized in the diagnostic and prognostic assays provided herein as well as the provided drug screening assays. Supernatants may also be used for purification and identification of the aging factors found within the supernatant that are responsible for the observed aged environment. Methods for purification and identification of said aging factors may be accomplished using methods well known to those skilled in the art. For recombinant expression of progerin in cells, e.g., neuronal progenitor cells or iPS cells, recombinant expression vectors comprising a progerin encoding nucleic acid may be generated.

Progerin is a truncated protein resulting from a specific lamin A mutation. As demonstrated herein, expression of progerin in neural progenitor cells leads to said cells exhibiting a robust AD phenotype. As used herein, the term “Progerin” refers to a protein having the amino acid sequence: of SEQ ID NO: 1. Also included are nucleic acids encoding the progerin protein of SEQ ID NO:1. Such nucleic acid sequences include, but are not limited to those nucleic acid sequences of SEQ ID NO:2. (See, Gene:LMNA CRNA title: mRNA-lamin A/C, transcript variant 7 Protein title: lamin isoform A-delta50 Protein comment: isoform A-delta50 is encoded by transcript variant 7 Protein ID: NP_001269555.1; ncbi.nlm.nih.gov/protein/NP_001269555.1)

The skilled artisan will readily appreciate that the embodiments are not limited to the progerin sequences depicted herein, but also include variants of progerin. Such variants may contain deletions, substitutions, or additions of one or more amino acids in the above depicted amino acid sequence of SEQ ID NO. 1 while maintaining the biological activity of progerin protein, e.g., the ability to promote AD phenotypes in cultured cells. As used herein, a peptide fragment or variant has amino acid sequences that are at least about 70-75%, typically at least about 80-85%, and most typically at least about 90-95%, 97%, 98% or 99% or more identity with the progerin protein (SEQ ID NO. 1) or peptide fragments thereof. In certain embodiments, a fragment or variant will contain conservative substitutions.

Methods for preparation of nucleic acid expression vectors are well established. See, e.g., Sambrook and Russell (2001), “Molecular Cloning: A Laboratory Manual,” 3rd ed, (CSHL Press). Many vectors are known in the art. Vector components may include one or more of the following: a signal sequence, an origin of replication, one or more selective marker genes (that may, for example, confer antibiotic or other drug resistance, complement auxotrophic deficiencies, or supply critical nutrients not available in the media), an enhancer element, a promoter, and a transcription termination sequence, all of which are well known in the art.

An “expression vector” or “expression construct” as used herein refers to a recombinant DNA molecule containing a desired coding sequence, e.g. progerin, and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto.

Methods well known to one of skill in the art can be used to construct expression vectors containing the coding sequence with appropriate transcriptional and translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989).

Typically, the vectors are derived from virus, plasmid, prokaryotic or eukaryotic chromosomal elements, or some combination thereof, and may optionally include at least one origin of replication, at least one site for insertion of heterologous nucleic acid, and at least one selectable marker. Embodiments are also contemplated that express progerin using artificial chromosomes, e.g., bacterial artificial chromosomes (BACs), yeast artificial chromosomes (YACs), mammalian artificial chromosomes (MACs), and human artificial chromosomes (HACs).

In such vectors, typically, a promoter region would be operably associated with a nucleic acid encoding progerin if the promoter was capable of effecting transcription of that nucleic acid. The promoter can be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit a-globin, as well as other sequences capable of controlling gene expression in eukaryotic cells. Additional suitable transcription control regions include tissue-specific promoters and enhancers as well as inducible promoters (e.g. promoters inducible tetracyclines). Such tissue specific promoters include those that function in neuronal cells.

In a specific embodiment, expression of progerin may be accomplished by introducing a recombinant virus carrying DNA or RNA encoding the progerin to one or more cells. Additionally, the recombinant virus may carry DNA or RNA encoding one or more familial AD mutations. Examples of recombinant viruses include, but are not limited to, retroviruses (including lentiviruses), adenoviruses, and adeno-associated viruses.

The term “host cell” means a cell that has been transformed, or is capable of being transformed, with a nucleic acid and thereby expresses a gene of interest. The polynucleotides encoding the progerin protein may be expressed in any appropriate host cell, preferably a mammalian cell. Exemplary mammalian host cells are neuronal progenitor cells or iPS cells. Other useful mammalian cell lines are well known and readily available from the American Type Culture Collection (ATCC) (Manassas, Va., USA) and the National Institute of General Medical Sciences (NIGMS) Human Genetic Cell Repository at the Coriell Cell Repositories (Camden, N.J., USA).

The compositions described above may be used in methods for diagnosing or prognosing AD in a subject. The term “subject” as used herein refers to an animal. Typically the animal is a mammal. In certain embodiments, the subject is a primate. In yet other embodiments, the subject is a human. A subject may be a subject that is suffering from AD or that has a risk factor for developing AD.

Accordingly, the present disclosure provides methods for diagnosing or prognosing AD in a subject comprising: (i) obtaining induced pluripotent stem cells derived from a subject wherein said cells have been recombinantly engineered to express progerin and subjected to cell culture conditions leading to neuronal differentiation; (ii) detecting one or more AD associated phenotypes in said cell culture; and (iii) determining that the subject has AD or is at risk of developing AD by detecting an increase in the level of AD associated phenotypes relative to a control biological sample.

In another embodiment, methods for diagnosing or prognosing AD in a subject are provided comprising the steps of: (i) obtaining induced pluripotent stem cells derived from the subject wherein said cells have been subjected to culture conditions leading to neuronal differentiation; (ii) contacting said cells with cell culture medium that has been derived from neuronal progenitor cells that exogenously express progerin and wherein said cell culture medium comprises secreted aging factors; and (ii) detecting one or more AD associated phenotypes in said cell culture.

The present disclosure provides methods for identifying a therapeutic agent that corrects a phenotype associated with development of AD comprising (i) contacting a first population of cells derived from neural progenitive cells expressing exogenous progerin, with a candidate therapeutic agent; (ii) contacting a second population of cells derived from neural progenitive cells expressing exogenous progerin with a control agent; (iii) assaying the two populations and (iv) identifying candidate agents as correcting the AD associated phenotype if the first population is closer to a normal phenotype following treatment than the second population.

iPS cell lines and iPS-derived cells generated from a subject (e.g., a human subject) can be used to determine the likelihood that a particular drug will have sufficient efficacy in that subject and, if so, an appropriate dose range for that subject. iPSC-derived cells from a subject, e.g., differentiated iPSC-derived cells may be exposed ex vivo to a drug to be tested, and then assayed for their phenotypic response to the drug as described herein. The response of the iPSC-derived cells may be compared to a reference response obtained in iPSC-derived cells from one or more individuals in which the drug has been shown to be effective and/or a reference response in iPSC-derived cells from subjects in which the drug was found to be ineffective. In some cases, the subject to be tested is a subject suffering from AD or having a predisposition to AD. For example, where the subject is suffering from AD, and multiple drugs are available to treat the AD, the efficacies and adverse effects of the multiple drugs may be evaluated using iPSC-derived cells from that individual. In other cases, the subject is not suffering from a health condition.

Accordingly, using iPS cells derived from a specific subject, a method is provided to identify or determine the likelihood that a particular therapeutic agent will have sufficient efficacy in that particular subject, and if so, an appropriate dose range for that subject. Accordingly, the present disclosure also provides methods for identifying a therapeutic agent that corrects a phenotype associated with development of AD in a specific subject comprising (i) contacting a first population of iPSC cells derived from said subject and expressing exogenous progerin, with a candidate therapeutic agent; (ii) contacting a second population of iPS cells derived from said subject and expressing exogenous progerin with a control agent; (iii) assaying the two populations and (iv) identifying candidate agents as correcting the AD associated phenotype if the first population is closer to a normal phenotype following treatment than the second population.

The present disclosure also provides methods for identifying a therapeutic agent that corrects a phenotype associated with development of AD in a specific subject comprising (i) contacting a first population of iPSC cells derived from said subject with a candidate therapeutic agent in the presence of cell culture medium that has been derived from neuronal progenitor cells that exogenously express progerin and wherein said cell culture medium comprises secreted aging factors; (ii) contacting a second population of of iPSC cells derived from said subject with a control agent in the presence of cell culture medium that has been derived from neuronal progenitor cells that exogenously express progerin and wherein said cell culture medium comprises secreted aging factors; (iii) assaying the two populations and (iv) identifying candidate agents as correcting the AD associated phenotype if the first population is closer to a normal phenotype following treatment than the second population.

In an embodiment, the neural progenitive cells, or iPS cells, for use in the disclosed diagnostic and screening methods comprise a familial AD mutation. In one aspect, the AD associated phenotype for both the diagnostic and drug screening assays includes, but is not limited to, increased tau phosphorylation, amyloid plaque accumulation, elevated levels of Aβ42 to A β42 ration, increased cell death and cell cycle re-entry.

The present disclosure relates to methods for identifying an agent that corrects a phenotype associated with development of AD comprising (i) contacting a first population of cells derived from neural progenitive cells expressing exogenous progerin, with a candidate agent; (ii) contacting a second population of cells derived from neural progenitive cells expressing exogenous progerin, with a control agent; wherein the cells in both populations comprise at least one familial AD mutation; (iii) assaying the two populations and (iv) identifying candidate agents as correcting the phenotype if the first population is closer to a normal phenotype following treatment than the second population.

“Therapeutic agent,” as used herein, refers to any test compound to be assayed for its ability to correct, reduce or alleviate the observed AD cell cultured phenotypes. “Correcting” a phenotype, as used herein, refers to altering a phenotype such that it more closely approximates a normal phenotype. Candidate therapeutic agents may be proteins, polypeptides, or individual small molecules. In some cases, the candidate therapeutic agents to be screened come from a combinatorial library, i.e., a collection of diverse chemical compounds generated by either chemical synthesis or biological synthesis. Preparation and screening of such combinatorial libraries are well known in the art.

In an embodiment, the culture phenotype associated with the development of AD (“AD biomarker”) is selected from the group consisting of increased tau phosphorylation, amyloid plaque accumulation, elevated levels of Aβ42 to Aβ40 ratio, increased cell death and cell cycle re-entry. Methods for detecting each of the AD associated phenotypes, i.e., AD biomarkers, are well known in the art.

In some embodiments, AD biomarker levels are measured by determining the gene expression of a given biomarker. In some embodiments, the expression level of the biomarker is determined by measuring the mRNA or miRNA level of the biomarker. In certain aspects, gene expression of the biomarker is determined using PCR, microarray, or sequencing. In some embodiments, the biomarker is measured by determining the level of protein expression. Such methods may be selected from the group consisting of immunohistochemistry, immunocytochemistry, immunofluorescence, immunoprecipitation, western blotting, and ELISA. These devices and methods can utilize labeled molecules in various sandwich, competitive, or non-competitive assay formats, to generate a signal that is related to the presence or amount of a polypeptide or protein of interest. Additionally, certain methods and devices, such as biosensors and optical immunoassays, may be employed to determine the presence or amount of polypeptide or protein without the need for a labeled molecule.

The analysis of a plurality of AD biomarkers may be carried out separately or simultaneously with one test sample. Several markers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in marker levels, as well as the absence of change in marker levels, would provide useful information about the disease status that includes, but is not limited to identifying the approximate time from onset of the event, the appropriateness of drug therapies, the effectiveness of various therapies, identification of the severity of the event, identification of the disease severity, and identification of the patient's outcome, including risk of future events.

Biomarkers of AD as disclosed herein serve an important role in the early detection and monitoring of AD. The measured amount can correlate to the presence or absence of AD, or the probability of developing AD in the future. In patients receiving treatment for their condition the measured amount will also correlate with responsiveness to therapy.

In one embodiment, the level of expression of Aβ40 and Aβ42 amyloids and their ratio of expression serves as a biomarker for AD wherein the Aβ42 and Aβ40 ratio is increased in cells expressing progerin. Levels of Aβ40 and Aβ42 amyloids may be detected using various different assay methods. In one aspect, an immunoassay, such as an ELISA assay is used to detect the levels of Aβ40 and Aβ42 amyloids. For example, kits are available for measuring Aβ42 and Aβ40 levels (Invitrogen).

One of the observed AD phenotypes associated with the expression of progerin is plaque formation. Such plaque formation may be monitored either using the filter retention assay or Congo red (CR) green birefringence. The filter retention assay separates Aβ plaques from soluble Aβ by retention on a filter membrane. Immunological detection of Aβ allows semiquantitative read outs. CR green birefringence is an anisotropic optical effect of CR-bound amyloid fibrils that can be visualized by polarizing microscopy. In another embodiment, Amylo-glo, a fluorescent histochemical probe may be used for the high resolution and contrast localization of amyloid plaques in tissue culture.

In another embodiment, detection of Tau phosphorylation may be used as a biomarker for AD where increased phosphorylation is indicative of AD. Detection of Tau phosphorylation in a sample may be accomplished using a variety of different assays including immunoassays using antibodies that bind specifically to phosphorylated Tau protein. For example, Western blots may be performed to assess the levels of Tau phosphorylation. In one embodiment, an antibody that recognizes Thr 231 phosphorylation is used. In other embodiments, antibodies that bind specifically to tau phosphorylated at S396, T181, Thr-153, Thr-181, Thr-205, Ser-199, Ser-202, Ser-214, Ser-235, Ser-262, Ser-356, Ser-396, Ser-422, Tyr18, Tyr29, Tyr197, Tyr310, and Tyr 394 may be used.

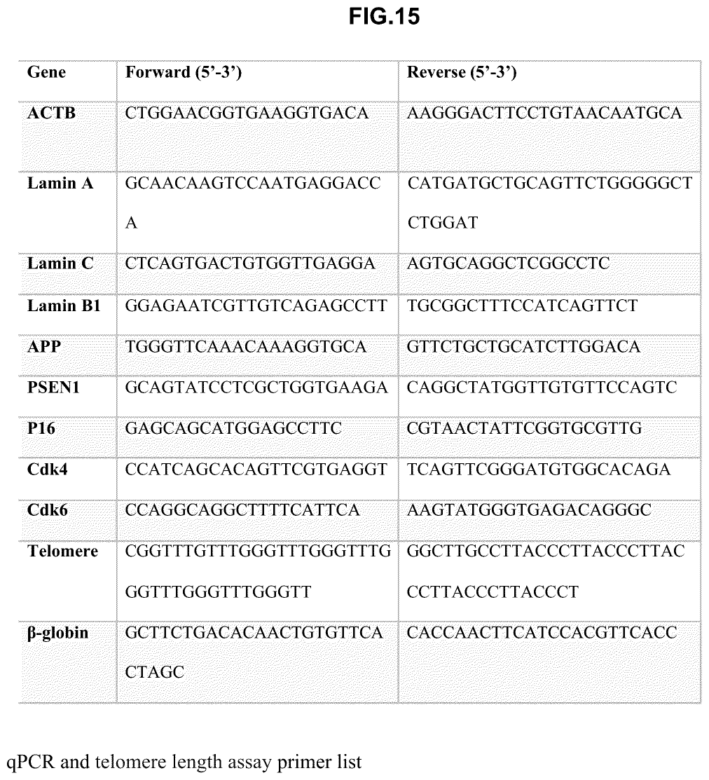

In addition, cell cycle re-entry and increased cell death are observed AD phenotypes in progerin expressing cells. Cell cycle reentry may be assayed in cells through detection of cell cycle markers such as p16 and cdk4/6. Detection of p16 and cdk4/6 may be achieved through assays designed to detect increased RNA or protein levels. FIG. 15 lists a number of different primers that may be used in PCR reactions to determine RNA expression levels. For detection of cell death, a number of different assays, well known to those of skill in the art, may be used including, for example, cell death flow cytometry may be used. Additionally, PI-annexin V apoptosis assays and cellular ROS assays may be used to detect cell death. For cell cycle re-entry, measurement of the incorporation of BrdU, which is a thymidine analog that labels the S phase of the cell cycle may be used. Cell death can be analyzed by propidium Iodide staining or using commercial assays such as Live/Dead from Thermo Fisher.

Pharmaceutical compositions of embodiments comprise a therapeutically effective amount of the therapeutic agents, identified using the screening methods disclosed herein, dissolved, or dispersed in a pharmaceutically acceptable carrier. The preparation of a pharmaceutical composition that contains at one or more of the identified therapeutic reagents and optionally an additional active ingredient will be known to those of skill in the art in light of the present disclosure, as exemplified by Remington's Pharmaceutical Sciences, 18th Ed. Mack Printing Company, 1990. For human administration, it will be understood that preparations should meet sterility, pyrogenicity, general safety and purity standards as required by FDA Office of Biological Standards or corresponding authorities in other countries. Preferred compositions are lyophilized formulations or aqueous solutions.

As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in therapeutic or pharmaceutical compositions is contemplated.

Any of the therapeutic agents, identified herein, may be used in therapeutic methods for treatment of AD. For use in the therapeutic methods described herein, such therapeutic agents would be formulated, dosed, and administered in a fashion consistent with good medical practice. Factors for consideration in this context include the particular disorder being treated, the particular subject being treated, the clinical condition of the subject, the cause of the disease or condition, the site of delivery of the agent, the method of administration, the scheduling of administration, and other factors known to medical practitioners or those of skill in the art.

The terms “treat,” “treating” or “treatment” of any disease or disorder as used herein refer in one embodiment, to halting the progression of the condition or disease, or to ameliorating the disease or disorder (i.e., slowing or arresting or reducing the development of the disease or at least one of the clinical symptoms thereof). In another embodiment “treat,” “treating” or “treatment” refers to alleviating or ameliorating at least one physical parameter including those which may not be discernible by the patient. In yet another embodiment, “treat,” “treating” or “treatment” refers to modulating the disease or disorder, either physically, (e.g., stabilization of a discernible symptom), physiologically, (e.g., stabilization of a physical parameter), or both. In yet another embodiment, “treat,” “treating” or “treatment” refers to preventing or delaying the onset or development or progression of the disease or disorder. As used herein, a subject is “in need of” a treatment if such subject would benefit biologically, medically or in quality of life from such treatment.

In certain embodiments the method further comprises administering to the individual a therapeutically effective amount of at least one additional therapeutic agent, e.g. an agent that is typically used to treat AD. A “subject” or “individual” according to any of the above embodiments is a mammal, preferably a human.

For the treatment of AD the appropriate dosage of the therapeutic agent (when used alone or in combination with one or more other additional therapeutic agents) will depend on the type of disease to be treated, the route of administration, the body weight of the patient, the severity and course of the disease, whether the therapeutic agent is administered for preventive or therapeutic purposes, previous or concurrent therapeutic interventions, the patient's clinical history and response to the therapeutic agent, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points.

The present disclosure also provides kits for diagnosing or prognosing AD in a subject, identifying a subject at risk of development of AD, or prescribing a therapeutic regimen or predicting benefit from therapy in a subject having AD, the kit comprising a component useful for detecting AD phenotypes in test cells. Such phenotypes include, for example, tau phosphorylation, amyloid plaque accumulation, elevated levels of Aβ42 to A β42 ration, increased cell death and cell cycle re-entry.

### Example 1

### Materials and Methods

hNPC cell culture. ReNcell VM immortalized human neural progenitor cells (hNPC) were purchased from EMD Millipore, initially derived from the ventral mesencephalon region of human fetal brains. Cells were expanded in cell culture plates coated with Matrigel (Corning) and grown in BrainPhys Neuronal Medium (STEMCELL) containing 20 ng/ml bFGF (R&D Systems), 20 ng/ml EGF (Millipore Sigma), 10 U/ml heparin (Sigma-Aldrich), B27 supplement (Thermo Fisher). For differentiation, cells were cultured in described medium lacking bFGF and EGF. For 3D cell culture on 4-chamber slides, 50 ul cold Matrigel was added to 50 ul cell suspension on ice. Matrigel/cell mixture was further diluted by adding 400 ul of the cold ReN differentiation medium and then seeded on chamber slides. After overnight 37° C. incubation, 200 ul of prewarmed ReN differentiation medium was added to each chamber.

Lentivirus packaging and transduction. HEK293T cells (ATCC) were co-transfected with lentiviral plasmids and two virus packaging vectors, psPAX2 and pMD2.G (Addgene), utilizing Fugene 6 (Promega). Culture supernatants were collected at 48 h and 72 h post-transfection, and filtered through 0.45 μm filters to remove any nonadherent 293T cells, then stored at −80° C. Next, ReN cells were infected by lentiviruses in media supplemented with Polybrene (Santa Cruz Biotechnology) with the final concentration of 8 g/ml. The medium was changed every other day post-infection until the cells were harvested. RNA isolation and quantitative PCR

Total genomic RNA was extracted with Trizol (Life Technologies, Carlsbad, CA. USA) and purified using the RNeasy Mini kit (Qiagen, Hilden, Germany) as per the manufacturer's instructions. The RNA yield was determined by the NanoDrop 2000 spectrophotometer (Thermo Fisher Scientific, Waltham, MA, USA). 600 ng of total RNA was converted to cDNA using the iScript Select cDNA Synthesis kit (Bio-Rad). Quantitative RT-PCR was performed in triplicates using SYBR Green Supermix (Bio-Rad) on the CFX96 Real-Time PCR Detection System (C1000 Thermal Cycler, Bio-Rad). All primers used in this study are listed in FIG. 15.

Genomic DNA extraction and quantitative PCR telomere assay. DNA samples were extracted from ReN cells with PureLink™ Genomic DNA Mini Kit (Invitrogen). The mean telomere length was assessed by the modified monochrome multiplex quantitative polymerase chain reaction method. Relative telomere length is shown as T/S ratio, which stands for the ratio of telomere repeat copy number to single copy gene copy number. All primers used in this study are listed in FIG. 15.

Western blot. Whole-cell lysates for immunoblotting were prepared by dissolving cells in Laemmli Sample Buffer containing 5%2-mercaptoethanol (Bio-Rad). Protein samples were loaded on 4-15% polyacrylamide gels (BioRad) and transferred onto 0.45 μm pore-size nitrocellulose membranes (Bio-Rad) using the Turboblot (BioRad). After that, blots were blocked with 5% milk for 1 h at room temperature. For phospho-tau, 5% BSA in TBS was used for blocking. Blots were incubated overnight at 4° C. with primary antibodies. And then blots were probed with secondary antibodies for 1 h at room temperature before ECL development and imaging (Bio-Rad). The primary antibodies used for immunoblotting are as follows: Lamin A/C antibody, (Abcam, 1:750); Lamin B1 antibody (Santa Cruz, 1:200); APP antibody (BioLegend, 1:400), total tau antibody (Santa Cruz, 1:200), PHF6 p-tau antibody (Santa Cruz, 1:200), γ-H2AX antibody (Abcam, 1:3000), and β-actin (1:5000, Sigma-Aldrich).

Cell cycle assay. Cells were harvested with accutase and then washed with PBS. Next, ice-cold 70% ethanol was added to the cells and then cells were incubated at 4° C. for 1 h. After PBS wash, samples were treated with RNase at 37° C. for 30 min to remove RNA content. 5 μg Propidium Iodide (Invitrogen) was added to the samples for another 30 min incubation at 37° C. Flow cytometry was performed with FACS CantoII (BD), and the data were analyzed by FlowJo software.

Cell death assay. PI-annexin V apoptosis assay was performed according to the manufacturer's instruction (Thermo Fisher, A35122). In brief, cells were harvested and rinsed with PBS and then resuspended and stained with 100 μL of 1× annexin V binding buffer, containing 5 μL of annexin V and 5 μL of PI, for 25 min in the dark at room temperature. Stained samples were analyzed by FACS CantoII (BD), and the data were processed by FlowJo software.

Oxidative stress assay. Cellular ROS Assay kit (Abcam, ab 186027) was used to check the oxidative stress according to the manufacturer's protocol. Cells were dissociated by accutase digestion, rinsed with PBS, and then incubated in 1=ROS Red Stock Solution for 30 min at 37° C. Flow cytometry was performed with FACS CantoII (BD), and the data were analyzed by FlowJo software.

ELISA. Aβ40 and Aβ42 levels were mainly measured by Invitrogen amyloid-β human ELISA Kit (Thermo Fisher, KHB3481 and KHB3441) as per the manufacturer's protocol. The conditioned media from ReN cells were collected and diluted by 1:3 or 1:9 with a dilution buffer provided by the manufacturer. A plate reader (Thermo Scientific) was used to quantify Aβ40 and Aβ42 ELISA signals.

Immunofluorescence staining. Cells were washed twice with PBS and then fixed in 4% paraformaldehyde (PFA) for 15 min at room temperature. After that, cells were permeabilized with 0.5% triton in PBS for 5 min at room temperature and then washed twice in TBS. Following blocking was done with 4% BSA in TBS for 1 h at room temperature. Samples were then incubated with primary antibodies in 4% BSA in TBS overnight at 4° C. Primary antibodies were rinsed off with 5 washes of TBS. Samples were incubated with secondary antibodies in 4% BSA in TBS for 1 h at room temperature protected from light before being washed 5 times in TBS. Primary antibodies used include: MAP2 antibody (Abcam, 1:1000); β-tubIII antibody (Abcam, 1:1000); GFAP antibody (Cell Signaling, 1:1000); Lamin A/C antibody, (Abcam, 1:500); Lamin B1 antibody (Santa Cruz, 1:200). Secondary antibodies include: Alexa Fluor 488 donkey anti-rabbit IgG (1:1000, Invitrogen), Alexa Fluor 594 donkey anti-rabbit IgG (1:1000, Invitrogen), Alexa Fluor 488 donkey anti-mouse IgG (1:1000, Invitrogen), Alexa Fluor 594 donkey anti-mouse IgG (1:1000, Invitrogen) and Alexa Fluor 644 donkey anti-rabbit IgG (1:1000, Invitrogen).

For BrdU staining, cells were incubated in a 10 uM BrdU (BD #550891) labeling medium for 12 h. Cells were then washed with PBS, fixed and permeabilized. Afterward, cells were denatured in 2N HCl for 40 min at room temperature. Next, samples were incubated in Alexa Fluor 647 anti-BrdU antibody solution (1:1000, Invitrogen #B35133) at 4° C. overnight. Samples were protected and stained in vectashield mounting medium with DAPI (Vector) sealed with a coverslip and stored in the dark. Fluorescence images were acquired with a Zeiss LSM 710 confocal microscope (Zeiss International, Oberkochen, Germany).

Amylo-glo staining. Cells were washed three times with 0.9% (wt/vol) NaCl solution. Following adding 100 μl of 0.05× Amylo-Glo working solution, 3D culture cells were incubated for 5 min at room temperature. And then, the staining solution was removed. 200 μl of 0.9% saline was added and followed with 5-min incubation. Samples were washed three times with ddH2O, and further washed with 0.9% (wt/vol) NaCl solution three times. Samples were protected in antifade vectashield mounting medium without DAPI (Vector) and stored in the dark. Fluorescence images were acquired with a Zeiss LSM 710 confocal microscope (Zeiss International, Oberkochen, Germany).

Calcium preparation and imaging. Intracellular calcium labeling was prepared using Fluo-4 AM (Thermo Fisher Scientific) following manufacturer-provided protocols. Briefly, a 1 mM stock solution was prepared in anhydrous DMSO. Cells were incubated in standard cell specific medium for 1 hour at 37° C. with luM Fluo-4 AM. After incubation cells were rinsed with DPBS and then the fresh, appropriate medium was added. Cell samples were then immediately used for imaging using 488 nm laser to excite the Fluo-4 AM dye.

Data analysis. Statistical analyses were performed using GraphPad Prism 7 software. Data were analyzed using unpaired Student's 1-test for two groups. One-way and two-way analysis of variance (ANOVA) followed by post hoc multiple comparisons were used to compare the means of three or more groups. All experiments were repeated at least three times, and the results are presented as the mean±SD. A p value<0.05 was considered significant. Asterisks indicate statistical difference as follows: n.s., not significant; *p<0.05; **p<0.01; ***p<0.001; ****p<0.0001.

### Results

### Different Nuclear Lamins are Regulated Differently During Neural Differentiation.

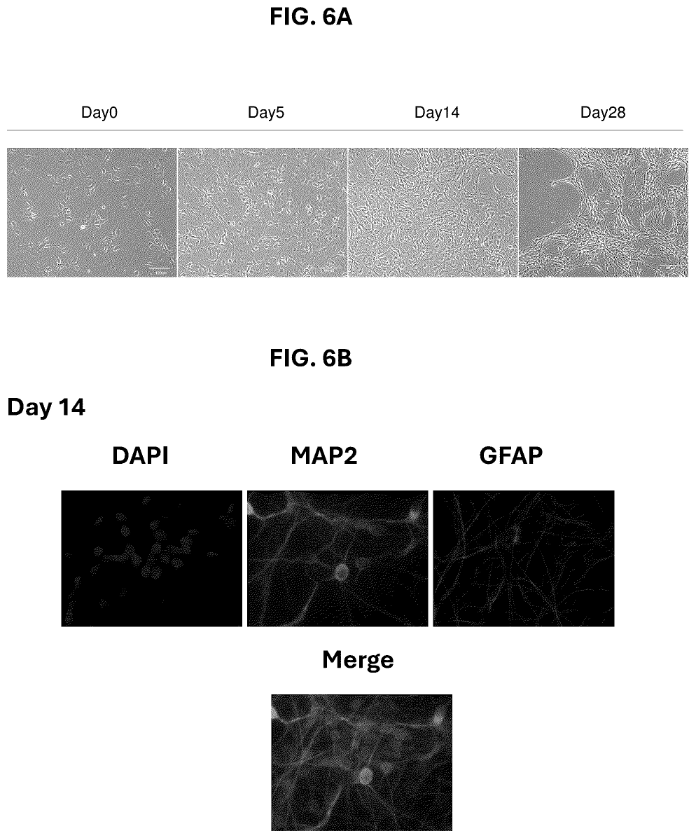

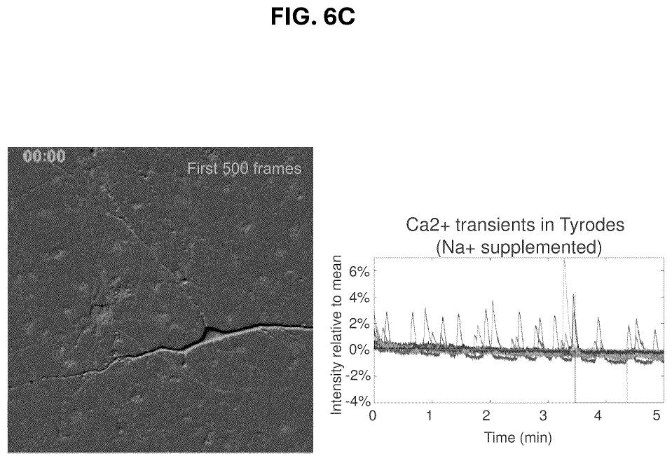

The expression of various lamins during the neural differentiation of ReN cells was first characterized using a commercial human neural progenitor cell (NPC) line (FIG. 6). By retracting growth factors from the medium, ReN cells generated neurite morphology (FIG. 6A). To validate the cell types, cells were detected with neuronal marker MAP2 and astrocytes marker GFAP by immunofluorescence staining. As expected, both markers were positively stained after two-week differentiation (FIG. 6B). To further determine if these cells were functional, the differentiated cells were stained with a Ca2+ indicator, Fluo-4. By measuring the fluorescence change, the Ca2+ transients could be detected and therefore reflect the action potentials (Grienberger, C. & Konnerth, A. Imaging Calcium in Neurons. Neuron 73, 862-885 (2012)). Cells at day 14 could generate Ca2+ transients regularly when the medium was replaced by Tyrode's solution supplemented with Na+(FIG. 6C). This result suggested that ReN cells could generate action potentials after differentiation.

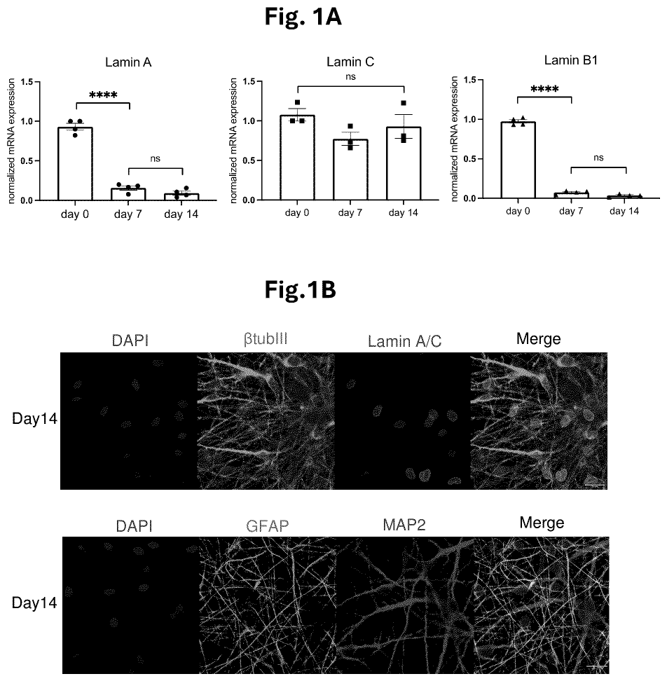

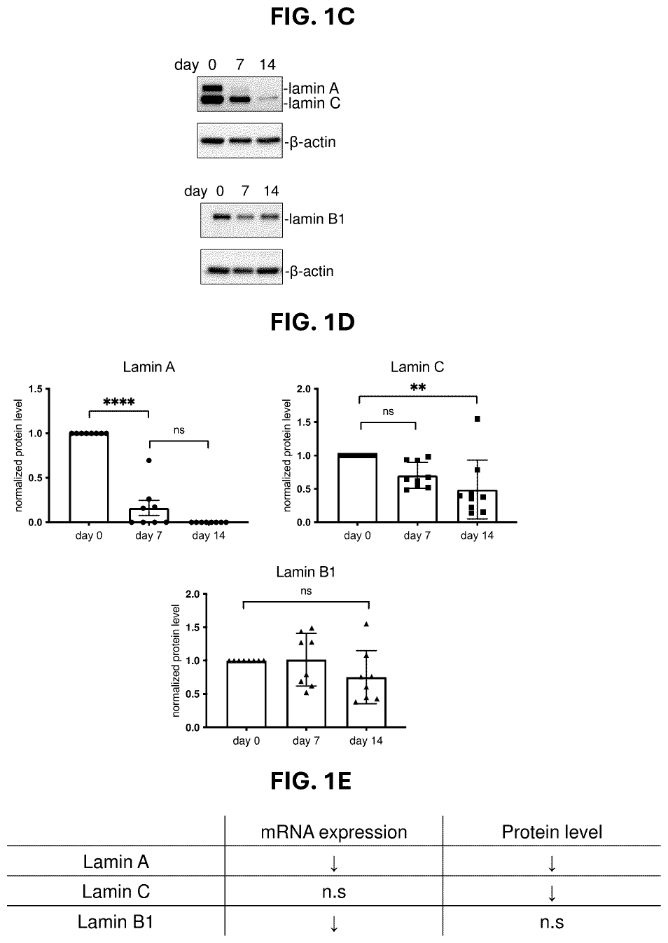

To determine the expression pattern of nuclear lamins in neurons, both transcriptional and translational expression of nuclear lamins was probed during differentiation (FIG. 1). It was found that at the mRNA level, both lamin A and lamin B1 were decreased, while lamin C did not show a significant change (FIG. 1A). However, at the protein level, lamin A and lamin C were downregulated, while lamin B1's protein level was relatively stable (FIG. 1C-D). These results (summarized in FIG. 1E) indicate that different nuclear lamins have different stability and different regulation mechanisms. While lamin A exists in the neural progenitor cells, it is diminished after the differentiation, which is consistent with previous findings (Jung, H. J. et al. Proc. Natl. Acad. Sci. U.S.A 109, 423-431 (2012)).

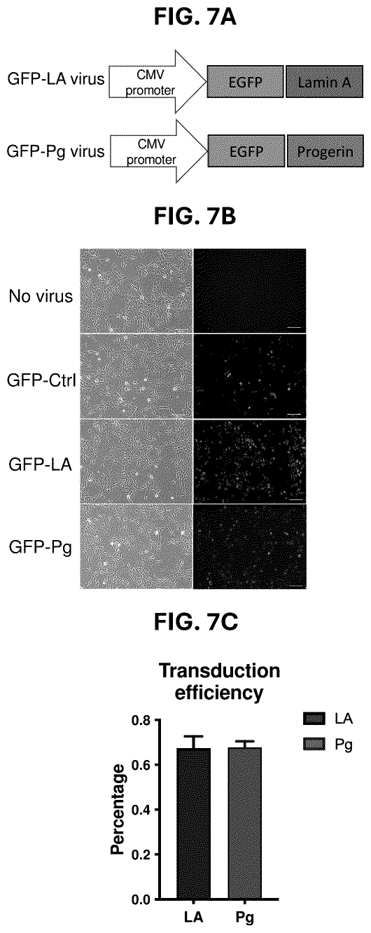

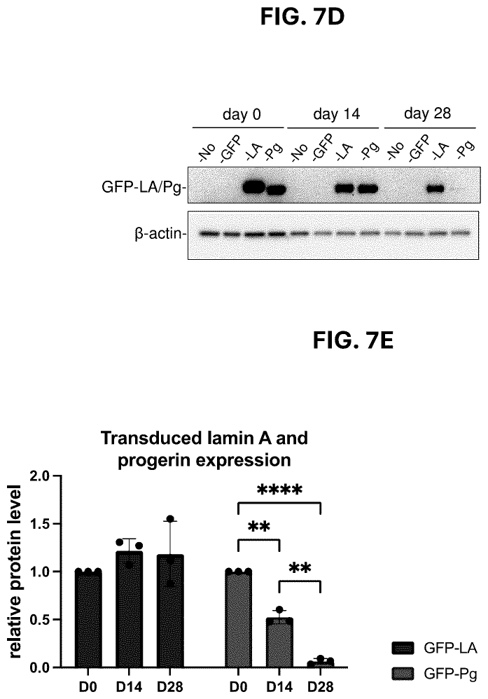

Overexpression of lamin A and progerin results in neural death and cell cycle re-entry. Healthy mature neural cells prefer expressing lamin C rather than lamin A (Jung, H. J. et al. Proc. Natl. Acad. Sci. U.S.A 109, 423-431 (2012)). However, abnormal lamin A accumulation has been observed in patients' hippocampus through the different stages of AD (Méndez-López, I. et al. Int. J. Mol. Sci. 20, (2019); Gil, L. et al. Int. J. Mol. Sci. 21, 1841 (2020)). To probe the potential role of A-type lamins in neurodegeneration, lamin A and progerin were overexpressed, respectively, in differentiating ReN cells to check their effects. Lentivirus constructs were used to express lamin A and progerin in ReN cells (FIG. 7A), as previously described (Wu, D., Yates, P. A., Zhang, H. & Cao, K Nucleus 7, 585-596 (2016)). Both lamin A and progerin were tagged with EGFP (Wu, D., Flannery, A. R., Cai, H., Ko, E. & Cao, K. Nucleus 7, 585-596 (2016). Flow cytometry analysis indicated that transduction efficiencies were 71.1% and 64.8% in lamin A and progerin, respectively (FIG. 7B-C). After transduction, ReN cells underwent the differentiation process. The exogenous protein expression during the differentiation was checked. By quantifying the protein level with Western blot (FIG. 7D-E), it was found that exogenous lamin A and progerin were still abundant after 2-week differentiation but exogenous progerin was decreased after 4 weeks. Thus, age-related phenotypes were detected at a 2-week timepoint.

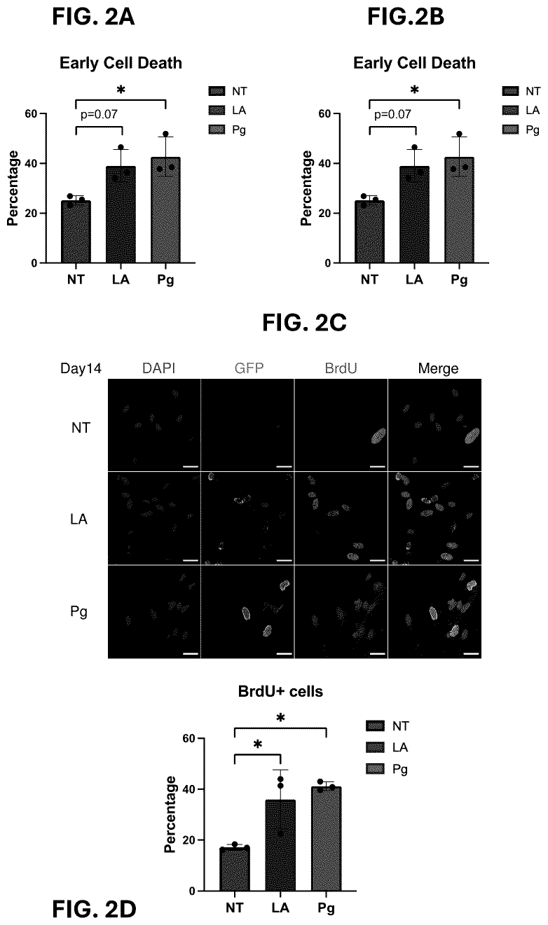

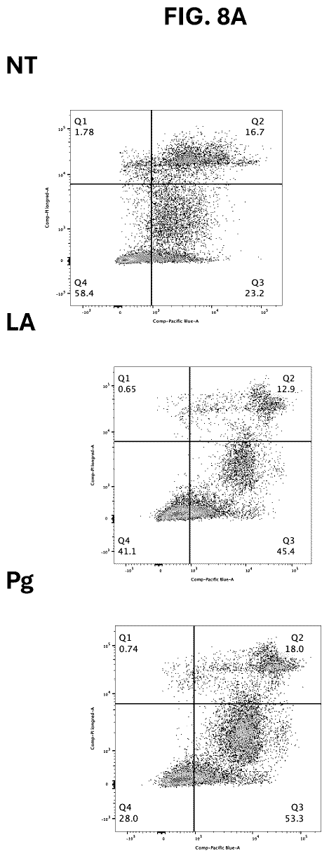

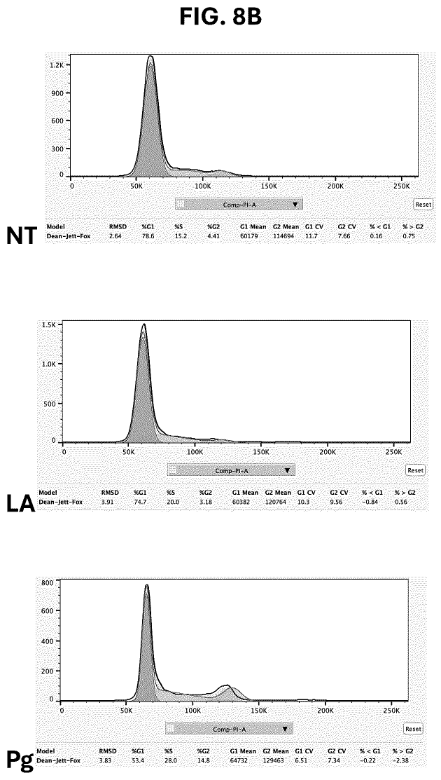

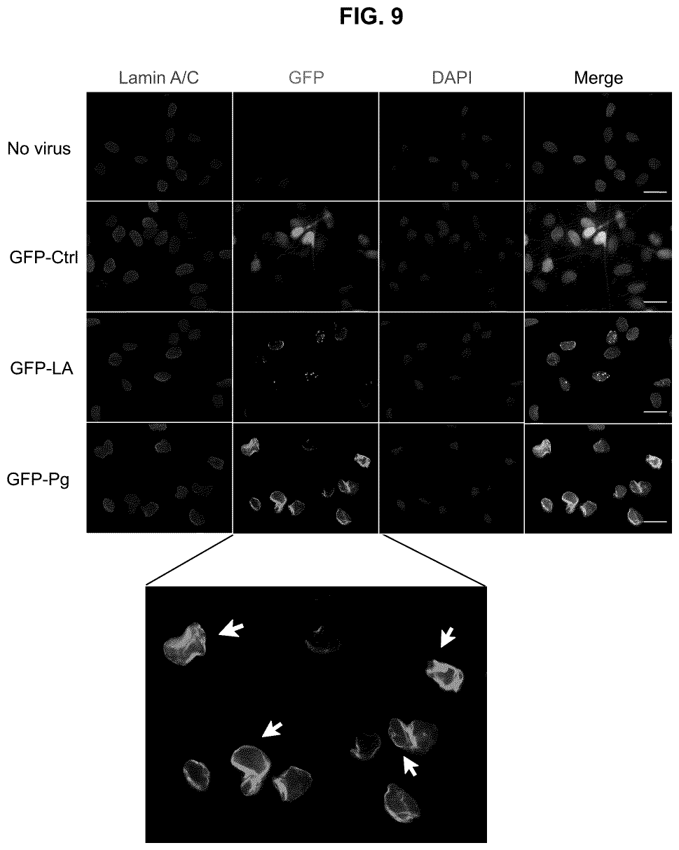

Cell death is a widely accepted feature of AD (Goel, P. et al. Front. Mol. Neurosci. 15, 1-20 (2022)), and cell death flow cytometry was performed to check this phenotype. After two weeks of differentiation, a slight increase in the proportion of early cell death events in cells expressing lamin A was observed compared to non-transduced cells (FIG. 2A, FIG. 8A). Moreover, progerin-transduced cells showed a significant increase in cell death compared to non-transduced cells at the same time point. Although neurons are known to quit the cell cycle and stay in the quiescent stage (GO), cell cycle re-entry is considered an early event in neurodegeneration and is related to cell death (Kruman, I. I. et al. Neuron 41, 549-561 (2004); Barrio-Alonso, E. et al. Sci. Rep. 8, 14316 (2018)). A significantly higher percentage of S-phase cells was detected after 2-week lamin A-transduction, compared to the cells without any transductions (FIG. 2B, FIG. 8B), which indicated increased cell cycle re-activation. The cells were also stained with BrdU to further validate the S-phase cells and confirmed that the percentage of S-phase cells was increased after 2 weeks (FIG. 2C-D). Nuclear morphology changes were observed after progerin expression but not after lamin A expression (FIG. 9), which indicated that progerin could disrupt the nucleoskeleton more drastically. In summary, the overexpression of lamin A led to heightened oxidative stress, cell death, and cell cycle re-entry, while progerin exacerbated these characteristics.

The combination of progerin and FAD mutations accelerated AD hallmark phenotypes. As stated previously, the age clock is reset in iPSC-derived neurons even if the cells are from old donors (Lapasset, L. et al. Genes Dev. 25, 2248-2253 (2011). In addition, in vitro cell culture often positively selects the more proliferative (i.e., younger and healthier) cells. Therefore, modeling an age-associated disease in vitro is tricky. Since it was found that ectopic lamin A- or progerin-expression could induce age-associated phenotypes in ReN cells after 2-week differentiation (FIG. 2), the question was asked if lamin A- or progerin-expression could accelerate neurodegeneration in ReN cells with FAD mutations.

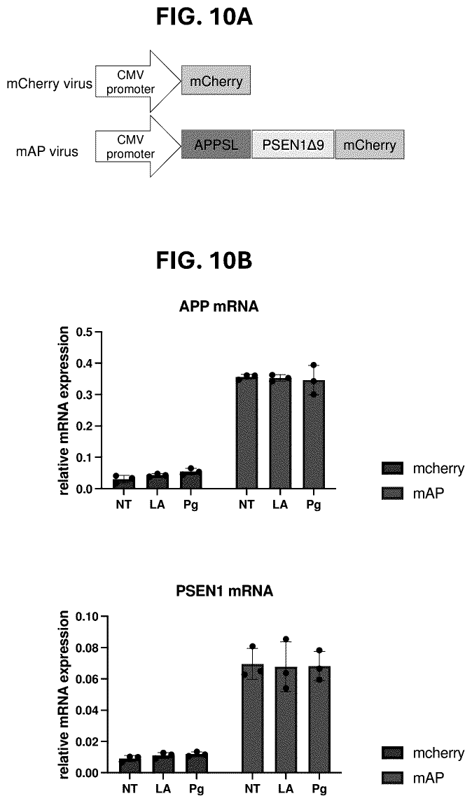

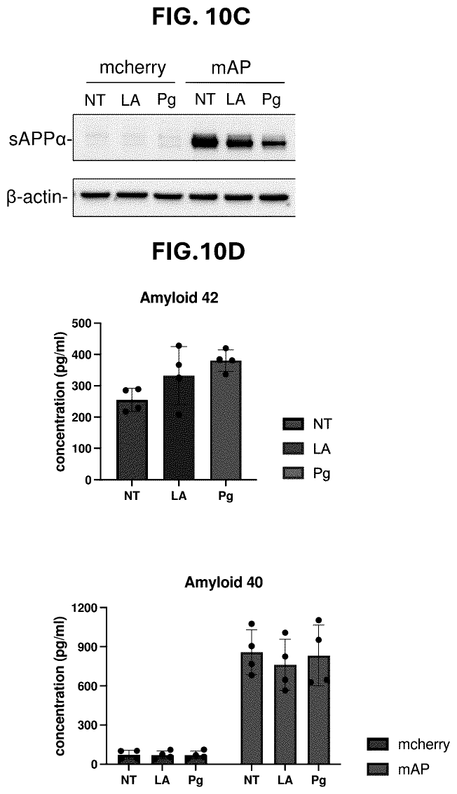

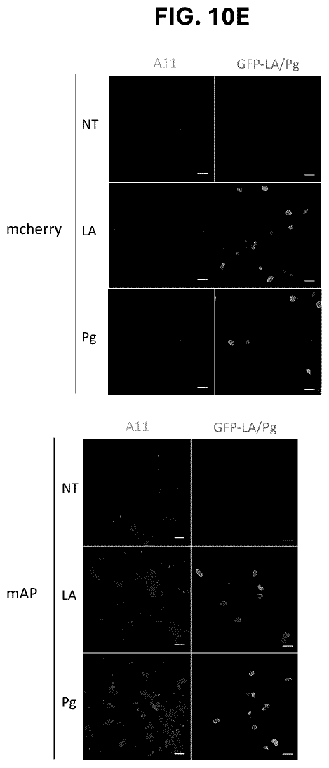

To test this idea, a well-characterized AD model (Kim, Y. H. et al. Nat. Protoc. 10, 985-1006 (2015)) was adapted, which is the first cell culture model recapitulating both Aβ plaques and tau aggregations. The plasmid containing APP with both the K670N/M671L (Swedish) and V717I (London) mutations (APPSL) and PSEN1 with the 49 mutation (PSEN1(49)) was utilized to introduce FAD mutants (FIG. 10A), as described (Kim, Y. H. et al. Nat. Protoc. 10, 985-1006 (2015)). The ReN cells were transduced with lentivirus containing mcherry-tagged APPSL and PSEN1(49) mutations (mAP) before the differentiation and mcherry-only lentivirus was used as the control group. For 2D culture, cells were seeded on the Matrigel-coated plates. For 3D culture, the cell suspension was mixed with Matrigel with a certain ratio and then plated on the chamber slides. After 3 days, both APP mRNA and PSEN1 mRNA were increased after mAP transduction (FIG. 10B). After a 2-week differentiation period, cells were transduced with either GFP-lamin A (LA) or GFP-progerin (Pg). Two days after transduction, both lamin A and progerin expression were robust. However, after 2 weeks, while lamin A expression remained strong, progerin expression became very weak (FIG. 10C).

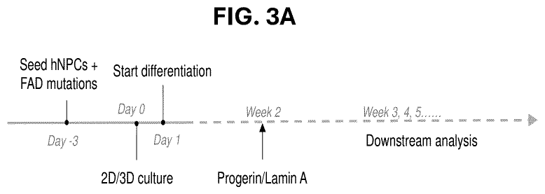

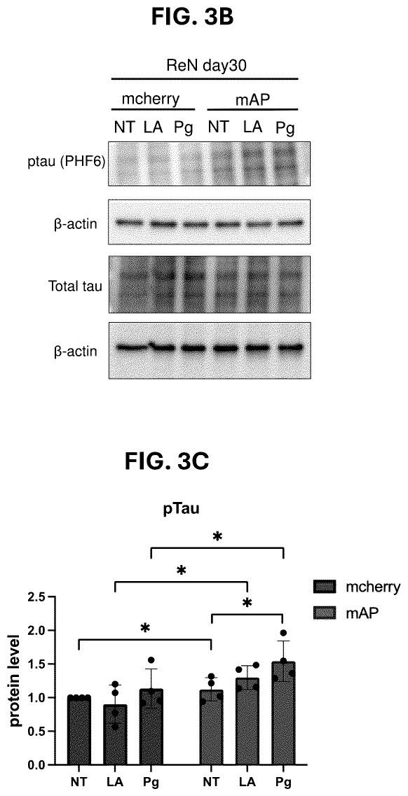

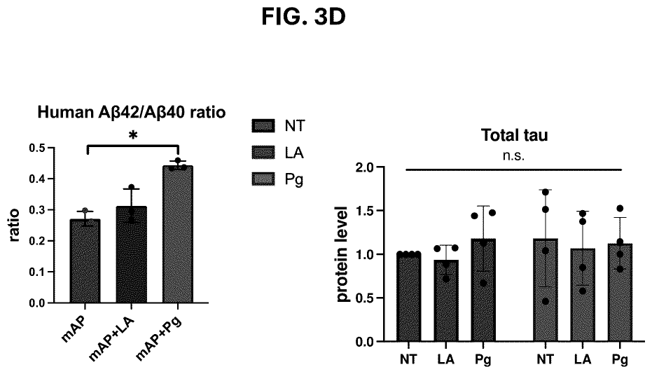

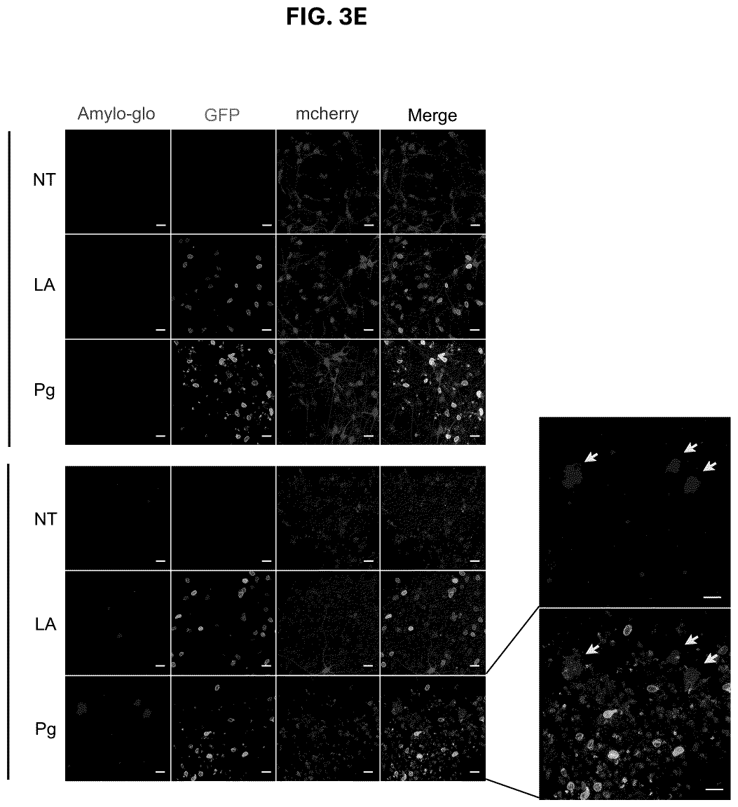

Tau phosphorylation is an important hallmark of AD (Kolarova, M. et al, Int. J. Alzheimers. Dis. 2012, 1-13 (2012)). Western Blot was used to check the protein level of total tau and phosphorylated tau weekly. It is reported that the phosphorylation of Thr231 tau is an early event in AD among different phosphorylation sites (Luna-Muñoz, J. et al. J. Alzheimer's Dis. 8, 29-41 (2005)). It is estimated that ptau at Thr231 contributes about 26% of the overall inhibition of tau binding to microtubules (Sengupta, A. et al. Arch. Biochem. Biophys. 357, 299-309 (1998)). Thus, an antibody that recognizes Thr231 phosphorylation site was used to detect tau phosphorylation. Although the total tau level was not significantly changed, ptau at Thr231 was slightly increased after lamin A expression compared to the cells with FAD mutations only, and progerin expression further upregulated tau phosphorylation significantly at week 4 (FIG. 3B-C).

Amyloid is another crucial pathology marker for AD (Chen, G. F. et al. Acta Pharmacol. Sin. 38, 1205-1235 (2017)). A higher Aβ42/Aβ40 ratio usually indicates more neurotoxicity, and it could be used as a more sensitive marker (Kuperstein, I. et al. Neurotoxicity of Alzheimer's disease Aβ peptides is induced by small changes in the Aβ42 to Aβ40 ratio. EMBO J. 29, 3408-3420 (2010)). ELISA was used to detect the Aβ40 concentration, Aβ42 concentration, and Aβ42/Aβ40 ratio in the culture medium after 3-week culture. Aβ40 level was upregulated in the mAP group after 3 weeks (FIG. 10D). Aβ42 was barely detectable in the mcherry control group but was again upregulated in the mAP group after 3 weeks (FIG. 10D). In the mAP group, the Aβ42/Aβ40 ratio showed a slight increase following the introduction of lamin A compared to cells carrying FAD mutants alone, and progerin expression significantly raised the Aβ42/Aβ40 ratio in comparison to cells with FAD mutants alone (FIG. 3D).

Next, Aβ aggregation formation was checked weekly in both 2D and 3D culture. In the 2D cell culture, the cells were checked with an Aβ oligomer antibody. In the mcherry group, Aβ oligomers were barely detected. In the mAP group, cells expressing both FAD mutations and progerin displayed stronger Aβ oligomer staining compared to cells expressing only FAD mutations after 3 weeks of culture (FIG. 10E). Aβ usually diffuses into media in 2D cell culture and therefore it is difficult to detect fibril formation. To further visualize the aggregation, a 3D cell culture with Matrigel was adapted, and Amylo-glo was used to detect the Aβ fibrils weekly. Amylo-glo signals were first detected in the mAP group after four weeks of culture, whereas the signal remained negative in the mcherry control group (FIG. 3E). Notably, larger Aβ fibrils were observed in the mAP cells expressing both progerin and FAD mutations after four weeks, as compared to the cells expressing FAD mutations alone (FIG. 3E). These results indicated that ReN cells with the combination of progerin and FAD mutants displayed accelerated disease phenotypes after only 3-4 weeks in both 2D and 3D cell culture, including tau phosphorylation and formation of β-amyloid.

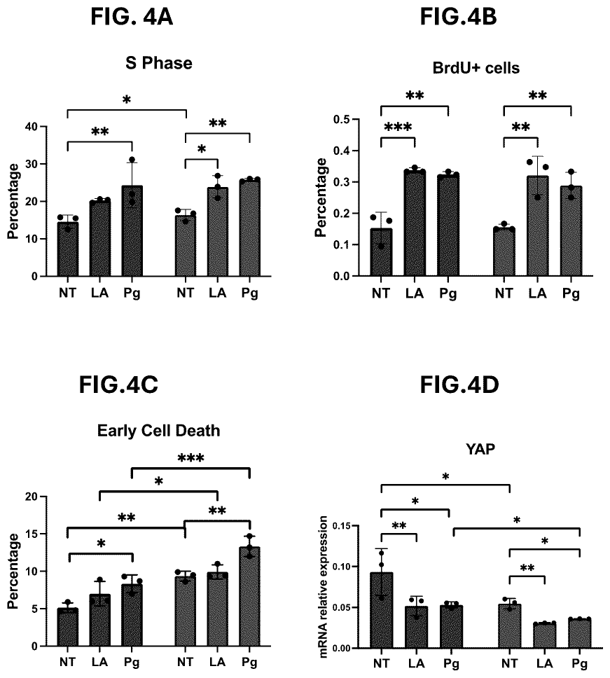

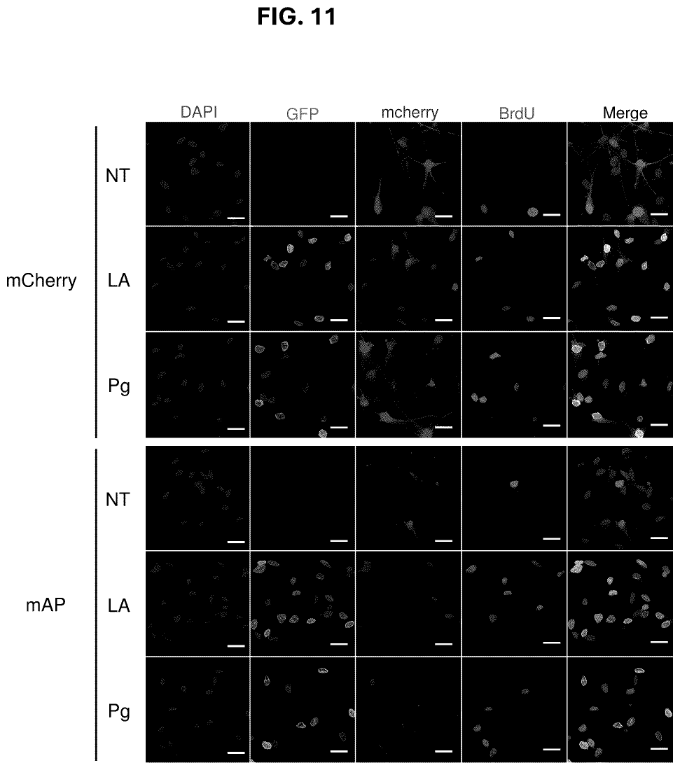

The combination of progerin and FAD mutations leads to increasing cell cycle re-entry and increasing cell death. After checking the AD pathological hallmarks, how cell cycle reactivation changed by adding lamin A or progerin was investigated. Overall, progerin addition resulted in more cell cycle re-entry events in 4 weeks (FIG. 4A). Within the mCherry group, ectopic expression of lamin A slightly increased the percentage of S-phase cells, and progerin could lead to a more drastic increase in S-phase cells after 4 weeks, compared to the cells with mCherry signal alone. Within the mAP group, cells exhibited a significantly higher percentage of S-phase cells after the ectopic expression of either lamin A or progerin. Meanwhile, a mild increasing percentage of S-phase cells was observed, comparing cells with FAD mutants alone to those with mCherry control plasmid. Cell cycle re-entry was further checked with BrdU staining (FIG. 4B, FIG. 11). The number of BrdU-positive cells was significantly increased, comparing cells carrying FAD mutations with progerin to cells carrying FAD mutations without progerin (FIG. 4B, FIG. 11). The same trend could be observed for the combination of lamin A and FAD mutations.

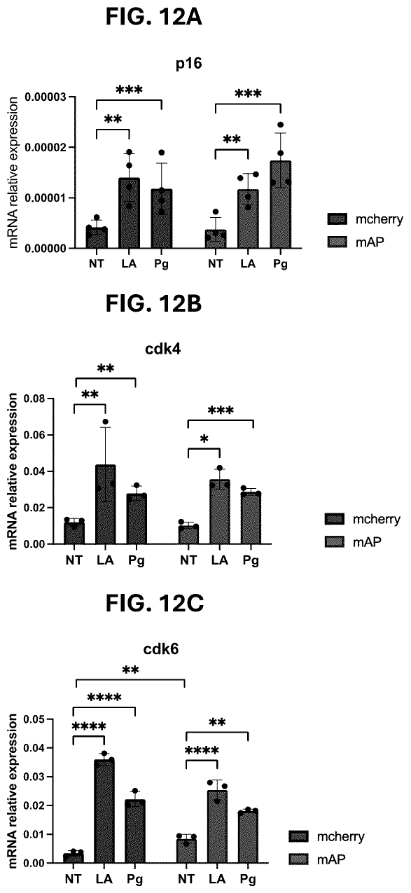

It is reported that increased p16 and cdk4/6 are associated with cell cycle dysregulation in AD (Mcshea, A. et al. Am. J. Pathol. 150, 1933 (1997); Bhat, R. et al. PLOS One 7, c45069 (2012)). Thus, the translational expression of these cell cycle regulators was investigated. In general, the intervention of progerin gave rise to the significantly elevated mRNA level of p16 and cdk4/6 in both mcherry control group and mAP group in 4 weeks (FIG. 12). Increased p16 level indicated that progerin could trigger senescence in cells and affect the environment. And cdk4/6 were involved in progerin-induced cell cycle reactivation. Ectopic expression of lamin A had the same trend (FIG. 12). Although the transcriptional expressions of p16 and cdk4 were indistinguishable between cells with FAD mutants and cells with mcherry control plasmid (FIG. 12A-B), cdk6 mRNA was significantly higher in cells with FAD mutants (FIG. 12C).

Since cell cycle re-entry has been associated with cell death in neural cells (Lee, H. gon et al. Neurochem. Int. 54, 84-88 (2009)), cell death flow cytometry was performed. Results showed that progerin addition significantly induced more cell death events in both mcherry control group and mAP group in 4 weeks (FIG. 4C). Comparing each parallel sample in mAP group to the sample in mcherry control group, significantly increased cell death was detected as well (FIG. 4C). These results indicated that progerin and FAD mutants could have a synergetic effect in cell death.

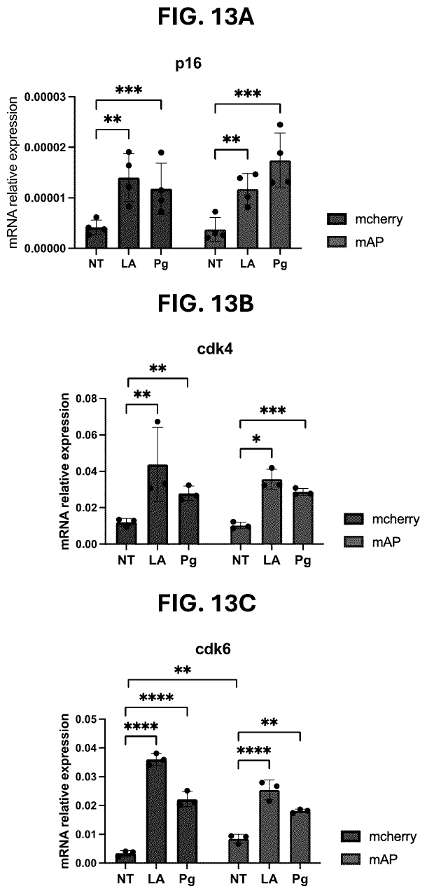

Cell death is a critical event in AD progression (Goel, P. et al. Front. Mol. Neurosci. 15, 1-20 (2022)) and the potential reasons in the disclosed system were checked. Yes-associated protein (YAP) is a critical factor of the Hippo signaling pathway, which responds to changes in cell mechanics (Piccolo, S., Dupont, S. & Cordenonsi, M. Physiol. Rev. 94, 1287-1312 (2014)). Several studies indicated that YAP was downregulated in AD brains (Xu, X. et al. Aging Cell 20, 1-16 (2021)) and it could be a critical regulator for cell death in AD (Tanaka, H. et al. Nat. Commun. 11, 1-22 (2020)). Here, the translational expression of YAP was checked. YAP mRNA was significantly downregulated in the cells with FAD mutants after 4 weeks, compared to the mcherry control group (FIG. 4D). The combination of progerin and FAD mutants exhibited a further reduction of YAP, compared to the cells with FAD mutants alone with 4-week culture (FIG. 4D). The accumulation of DNA damage could also contribute to cell death in AD (Madabhushi, R., Pan, L. & Tsai, L. H. Neuron 83, 266-282 (2014)). Here, γ-H2AX expression was checked as a marker for DNA damage. Ectopic expression of progerin could significantly upregulated γ-H2AX expression in both mAP group and mcherry control group in 4 weeks, whereas the difference between mcherry control group and mAP group was barely observed (FIG. 13A-B). One explanation is that cells with FAD mutants alone might not be aged enough to develop detectable DNA damage. Meanwhile, it is reported that progerin could have a synergistic connection with telomere damage in cellular senescence (Cao, K. et al. J. Clin. Invest. 121, 2833-2844 (2011)).

Here, the telomere length in both mcherry group and mAP group was checked. Telomere length was maintained after progerin overexpression (FIG. 13C), which indicated progerin-induced aging in neurons is independent of telomere damage.

Starting from the nuclear lamina, via the trans-membrane LINC complex to the cytoskeleton filaments, the nucleoskeleton and the cytoskeleton form a network of physically interconnected cellular components (Houben, F. et al. Biochim. Biophys. Acta-Mol. Cell Res. 1773, 675-686 (2007). The nuclear lamina plays a vital role in the signal transmission between the extracellular environment, cytoplasm, and nucleus (Gerace, L. & Tapia, O. Curr. Opin. Cell Biol. 52, 14-21 (2018)). During the aging process, age-related pathogenesis may occur due to changes in cellular mechanical properties resulting from disrupting the nucleocytoskeleton's integrity. This, in turn, could lead to dysfunctional changes. Protein aggregation is a common feature in most neurodegenerative diseases and could be linked to the disturbance in cell mechanics that occurs during the aging process. Meanwhile, laminopathies are mainly caused by mutations in the LMNA gene and manifest nuclear architecture disruption (Worman, H. J. J. Pathol. 226, 316-325 (2012)). One of the laminopathies is HGPS, a premature aging disease (Eriksson, M. et al. Nature 423, 293-298 (2003)). Observations have revealed several similarities between premature aging diseases and physiological aging. These similarities include instability in both genomic and proteomic structures, an increase in oxidative stress, and impaired DNA repair mechanisms (Kubben, N. & Misteli, T. Nat. Rev. Mol. Cell Biol. 18, 595-609 (2017)). Additionally, in a drosophila model of AD, it was indicated that disruption of lamin led to relaxation of heterochromatin, activation of the cell cycle, and ultimately, cell death, all of which contribute to neurodegeneration (Frost, B., Bardai, F. H. & Feany, M. B. Curr. Biol. 26, 129-136 (2016). Furthermore, numerous studies have demonstrated alterations in nuclear morphology and increased lamin A in individuals with Alzheimer's disease (Sheffield, L. G. et al. J. Neuropathol. Exp. Neurol. 65, 45-54 (2006); Frost, B. et al. Nat. Neurosci. 17, 357-366 (2014); Méndez-López, I. et al. Int. J. Mol. Sci. 20, (2019); Gil, L. et al. Int. J. Mol. Sci. 21, 1841 (2020) . . . . One group also observed reduced ZMPSTE24, a protein that plays a major role in the cleavage of the farnesylated tail in prelamin A, in patient's brain (Rosene, M. J. et al. Alzheimers. Dement. 17, e054396 (2021)). These findings indicated that disrupted nuclear lamina could participate in AD pathology and farnesylated lamin A is associated.

The study disclosed herein revealed that overexpression of lamin A in ReN cells resulted in elevated oxidative stress, reactivation of the cell cycle, and ultimately cell death. The presence of progerin, in particular, exacerbated these phenotypes, highlighting the role of lamin A in the development of AD pathology. As oxidative stress, cell cycle re-entry, and cell death are all crucial events in the aging process, these results indicate that the expression of lamin A or progerin may create an aging microenvironment conducive to the development of disease.

Currently, one of the biggest challenges in studying AD is accurately and efficiently modeling the disease. Considering the significant sequence differences in Aβ and tau between mice and humans, human tissue and cells could provide more accurate information (Xu, G. et al. Acta Neuropathol. Commun. 3, 72 (2015); Duff, K. et al. Neurobiol. Dis. 7, 87-98 (2000)) 38. However, a significant limitation in creating representative patient-derived models is the insufficient availability of high-quality post-mortem tissue. Consequently, most human-based models rely on induced pluripotent stem cells (iPSCs) (Penney, J., Ralvenius, W. T. & Tsai, L.-H. Mol. Psychiatry 25, 148-167 (2020)). Nonetheless, there are currently no standardized protocols for generating and maintaining these cell lines, and even in 3D cultures, it takes several months to observe AD phenotypes (Kim, Y. H. et al. Nat. Protoc. 10, 985-1006 (2015)). Additionally, a concern with iPSC-derived models is that the physiological age of the iPSCs may be reset, while AD is a late-onset disease. Thus, generating aged cells to study AD has become a pressing issue. Increased lamin A in AD patients might retain the farnesylated tail, given the observation that ZMPSTE24 is downregulated in AD brains (Rosene, M. J. et al. Alzheimers. Dement. 17, e054396 (2021)) And progerin is the permanently farnesylated lamin A because of the loss of the ZMPSTE24 cleavage site (Eriksson, M. et al. Nature 423, 293-298 (2003)). Moreover, HGPS exhibits molecular characteristics similar to those of natural aging, making it an effective model for aging research. Thus, using progerin to disrupt nuclear architecture could serve as a valuable strategy for emulating an aging environment. Progerin was already shown to induce age-related phenotypes in iPSC-derived neurons, successfully reproducing disease-specific phenotypes in Parkinson's Disease (PD) (Miller, J. D. et al. Cell Stem Cell 13, 691-705 (2013)). Ectopic expression of progerin was also applied in Huntington's Disease (HD) and HD-associated gene profile changes were enhanced (Cohen-Carmon, D. et al. Mol. Neurobiol. 57, 1768-1777).

Here, it is possible that lamin A or progerin expression could create an environment conducive to the development of AD pathology. To test this hypothesis, exogenous lamin A or progerin was introduced into ReN cells containing FAD mutations to determine if AD-related features could be amplified. The results demonstrated that ReN cells containing both progerin and FAD mutations exhibited a significant increase in the Aβ42/Aβ40 ratio and tau phosphorylation within just 3-4 weeks (FIG. 3). Additionally, Aβ aggregation was checked, and stronger Aβ oligomer staining (FIG. 9) and more Aβ fibril formation was observed (FIG. 3E) in these cells after a 4-week culture period. It was also found that ectopic expression of either lamin A or progerin increased cell cycle re-entry events (FIG. 4A-B) and promoted cdk4/6 expression (FIG. 12B-C), which are important cell cycle regulators. Furthermore, a significant increase in cell death after progerin expression was noticed (FIG. 4C), which are crucial events in neurodegeneration. Increased cell death could be an explanation for decreased progerin in 2 weeks after progerin transduction (FIG. 10C). Meanwhile, the senescence marker p16 was examined and it was observed that both lamin A and progerin expression could significantly increase the mRNA level of p16, which supported that disrupted nucleoskeleton promoted senescence in cells and provided an aged environment for disease development (FIG. 12A). Additionally, the presence of p16 indicated inappropriate regulation of neuronal cell cycle. Increased p16 level could be a response to cell cycle reactivation.

In summary, only Aβ accumulations, increased tau phosphorylation and more cell death were detected with the combination of ectopic progerin expression and FAD mutations. These findings indicate that progerin expression could accelerate the progression of AD-related phenotypes. No significant difference in cell cycle re-entry was observed between mcherry control group and mAP group. Considering AD is a late-onset disease, 4-week expression of FAD mutations might not be able to induce distinct changes in cell cycle dysregulation. Therefore, utilizing progerin strategy to facilitate AD progression is necessary.

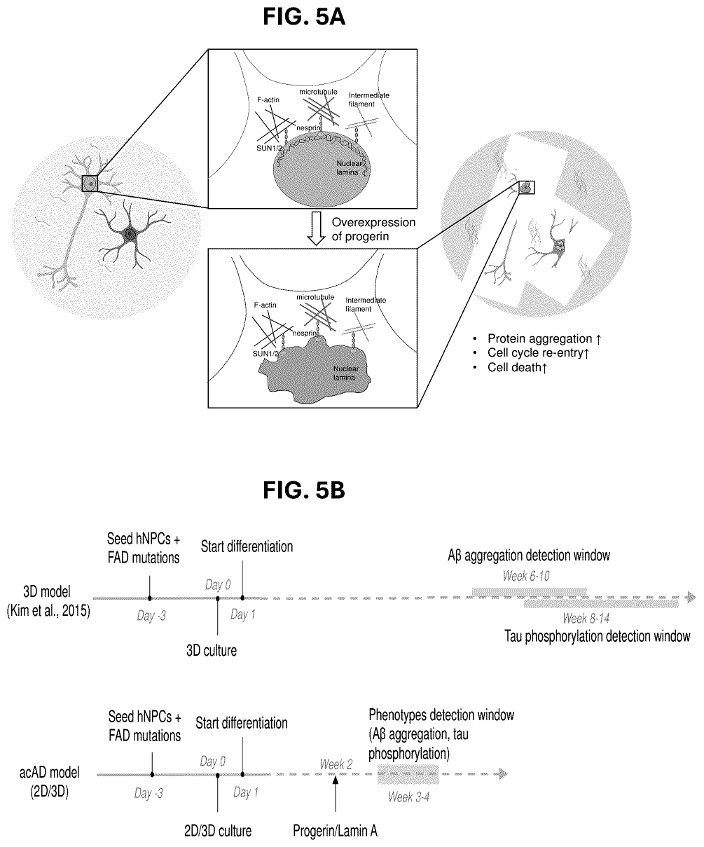

It is possible that cells containing only FAD mutations are not typically subject to aging in most circumstances. Therefore, their cell mechanics remain intact so they can clear toxic proteins. However, the balance of the nucleoskeleton is disrupted after progerin overexpression and progerin induces senescence in cells. And therefore, it provides a stiff and aged microenvironment for neighboring cells, making cells more vulnerable and leading to increased protein aggregation, cell cycle re-entry, and cell death (FIG. 5A). It has been reported that progerin expression induces senescence in human endothelial cells and promotes the secretion of senescent-associated secretory factors (SAPSs) (Bidault, G. et al. Cells 9, 1201 (2020); Xu, Q. et al. Eur. Hear. J. Open 2, 1-16 (2022), which in turn affects the neighboring cells and creates an aged endothelial cell culture environment. Such data provides possible evidence for a role of progerin in development of vascular diseases. In the present instance, it is believed that the introduction of progerin to the accelerated AD model system, as described herein, acts both cell autonomously and non-autonomously, to induce aging and accelerate neurodegeneration.

Compared to the time-consuming animal models and rejuvenated iPSC-derived models, the system disclosed herein serves as a more efficient model. This system was built on the well-characterized, leading 3D AD cellular model (Kim, Y. H. et al. Nat. Protoc. 10, 985-1006 (2015)), and therefore it was adapted to their timeline for comparison (FIG. 5B). The model disclosed herein demonstrates an accelerated manifestation of both increased tau phosphorylation and fibril formation within a significantly shorter timeframe of 4 weeks, in contrast to the traditional AD cellular models which typically require several months of experimental time. Meanwhile, traditional monolayer cell culture models fail to mimic the true brain architecture, resulting in Aβ diffusion in the culture medium (Centeno, E. G. Z., Cimarosti, H. & Bithell, A. Mol. Neurodegener. 13, 1-15 (2018)). The presently provided system can be utilized in both 2D and 3D cultures, providing the ability to easily adjust cell density and extracellular matrix thickness for a variety of analyses. Consequently, this accelerated AD model may be a more feasible platform for drug screening and investigating AD mechanisms, including neuronal death.

To summarize, the presently disclosed results establish a link between lamin A and AD pathology. It has been demonstrated that by inducing progerin expression in FAD-mutant cells, one can create an aged environment and generate strong AD features in a short amount of time (FIG. 5). This accelerated AD model can be used as an effective tool for AD research. Furthermore, this progerin-induced aging can be used as a general approach for modeling other late-onset diseases.

### Example 2

As disclosed herein, progerin-positive cells were decreased by week 4, the time of analysis. However, robust AD phenotypes were observed at this time. Additionally, the progerin transfection was not 100%, yet a system-wide increase in AD phenotypes was observed. For example, an increase in BrdU+ cells in immunofluorescent studies were observed, but many of them were progerin-negative. This led to the hypothesis that the progerin-transfected cells were modulating the entire cell environment and not just working intracellularly on the transfected cells.

In cellular senescence, cells are known to secrete senescence-associated secretory phenotype (SASP) factors, which generate an aged environment for all the cells in their vicinity. This cell-cell communication could account for the system-wide effects observed in the disclosed system, modulating even healthy cells and persisting even after progerin-positive cells die.

To test this hypothesis, a conditioned media experiment was performed. By taking media exposed to progerin-positive cells and transferring it to AD mutant-positive cells, it is possible to separate out the effects of progerin intracellularly versus intercellularly. If successful, it should be possible to isolate the most important SASP factors and build a highly efficient, aged model using SASP-loaded media to better recapitulate AD.

### Materials and Methods

Three days before the experiment, a neural progenitor cell line (ReN cells) was transfected with familial AD mutations in APP and PSEN1 (ReN+fAD). On day 0, three wells were seeded with non-transduced ReN cells, and three with ReN+fAD. The non-transduced ReN cells were transfected with no virus, lamin A, or progerin by a lentiviral vector on day 1 of differentiation. All cells were differentiated until day 7 without conditioned media treatment, with 50% media changes every 2-3 days. On day 8, and then every 2-3 days, the media changes for the fAD cells were switched to 50% conditioned media from the progerin-transduced cells or controls, and 50% fresh media. Conditioned media was centrifuged for 3 mins at 21,000×g before adding to the fAD cells in order to clear dead cells and debris. After 3 weeks of conditioned media treatment (4 weeks of differentiation), cells were collected, and qPCR and Western blot were performed.

### Results

In previous experiments, the number of progerin-expressing cells decreased by week 4 due to senescence. However, robust AD phenotypes were observed at this time.

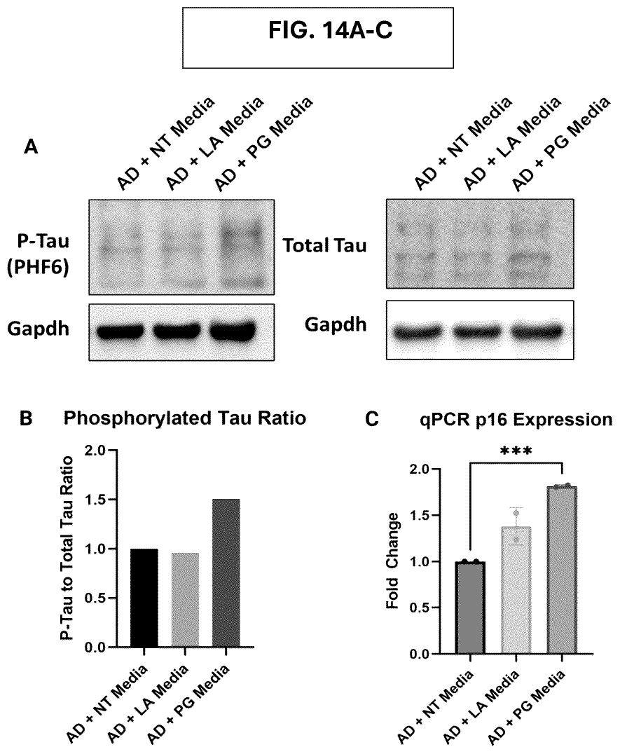

Additionally, the efficiency of progerin viral transduction was only 60-70%, yet an increase in AD-like phenotypes was observed (Tau phosphorylation and cell cycle re-entry) even in progerin-negative cells. This led to the hypothesisthat the progerin-transfected cells modulate the entire cell environment and not just work autonomously. Cell-cell communication and SASP factors could account for the system-wide effects in acAD, affecting neighboring cells and persisting even after progerin-positive cells die. A conditioned media experiment was then performed (FIG. 14). By taking the culture medium exposed to progerin-positive cells and transferring it to NPCs carrying FAD mutations, the effects of progerin intracellularly versus intercellularly can be separated. It was found that after 2 weeks of conditioned media treatment, progerin-conditioned media-treated FAD cells (AD+PG Media) showed a clear increase in Tau phosphorylation. In addition, AD+PG samples are significantly more senescent than AD+LA control and AD+NT mock control, as determined by p16 expression. These demonstrate that progerin-expressing neural cells modulate the entire cell environment non-autonomously by releasing phenotype-accelerating “aging factors.”

| SEQ ID NO: 1 | |
|---|---|
| ″METPSQRRATRSGAQASSTPLSPTRITRLQEKEDLQELNDRLAV | |
| YIDRVRSLETENAGLRLRITESEEVVSREVSGIKAAYEAELGDARKTLDSVAKERARL | |
| QLELSKVREEFKELKARNIKKEGDLIAAQARLKDLEALLNSKEAALSTALSEKRTLEG | |
| ELHDLRGQVAKLEAALGEAKKQLQDEMLRRVDAENRLQTMKEELDFQKNIYSEELRET | |
| KRRHETRLVEIDNGKOREFESRLADALQELRAQHEDQVEQYKKELEKTYSAKLDNARQ | |
| SAERNSNLVGAAHEELQQSRIRIDSLSAQLSQLQKOLAAKEAKLRDLEDSLARERDTS | |
| RRLLAEKEREMAEMRARMQQQLDEYQELLDIKLALDMEIHAYRKLLEGEEERLRLSPS | |
| PTSQRSRGRASSHSSQTQGGGSVTKKRKLESTESRSSFSQHARTSGRVAVEEVDEEGK | |
| FVRLRNKSNEDQSMGNWQIKRONGDDPLLTYRFPPKFTLKAGQVVTIWAAGAGATHSP | |
| PTDLVWKAQNTWGCGNSLRTALINSTGEEVAMRKLVRSVTVVEDDEDEDGDDLLHHHH | |
| GSHCSSSGDPAEYNLRSRTVLCGTCGQPADKASASGSGAQSPQNCSIM″ | |
| SEQ ID NO: 2 | |
| 1 | gagcgccgca cctacaccag ccaacccaga tcccgaggtc cgacagcgcc cggcccagat |
| 61 | ccccacgcct gccaggagca agccgagagc cagccggccg gcgcactccg actccgagca |
| 121 | gtctctgtcc ttcgacccga gccccgcgcc ctttccggga cccctgcccc gcgggcagcg |
| 181 | ctgccaacct gccggccatg gagaccccgt cccagcggcg cgccacccgc agcggggcgc |
| 241 | aggccagctc cactccgctg tcgcccaccc gcatcacccg gctgcaggag aaggaggacc |
| 301 | tgcaggagct caatgatcgc ttggcggtct acatcgaccg tgtgcgctcg ctggaaacgg |
| 361 | agaacgcagg gctgcgcctt cgcatcaccg agtctgaaga ggtggtcagc cgcgaggtgt |
| 421 | ccggcatcaa ggccgcctac gaggccgagc tcggggatgc ccgcaagacc cttgactcag |
| 481 | tagccaagga gcgcgcccgc ctgcagctgg agctgagcaa agtgcgtgag gagtttaagg |
| 541 | agctgaaagc gcgcaatacc aagaaggagg gtgacctgat agctgctcag gctcggctga |
| 601 | aggacctgga ggctctgctg aactccaagg aggccgcact gagcactgct ctcagtgaga |
| 661 | agcgcacgct ggagggcgag ctgcatgatc tgcggggcca ggtggccaag cttgaggcag |
| 721 | ccctaggtga ggccaagaag caacttcagg atgagatgct gcggcgggtg gatgctgaga |
| 781 | acaggctgca gaccatgaag gaggaactgg acttccagaa gaacatctac agtgaggagc |
| 841 | tgcgtgagac caagcgccgt catgagaccc gactggtgga gattgacaat gggaagcagc |
| 901 | gtgagtttga gagccggctg gcggatgcgc tgcaggaact gcgggcccag catgaggacc |
| 961 | aggtggagca gtataagaag gagctggaga agacttattc tgccaagctg gacaatgcca |
| 1021 | ggcagtctgc tgagaggaac agcaacctgg tgggggctgc ccacgaggag ctgcagcagt |
| 1081 | cgcgcatccg catcgacagc ctctctgccc agctcagcca gctccagaag cagctggcag |
| 1141 | ccaaggaggc gaagcttcga gacctggagg actcactggc ccgtgagcgg gacaccagcc |
| 1201 | ggcggctgct ggcggaaaag gagcgggaga tggccgagat gcgggcaagg atgcagcagc |
| 1261 | agctggacga gtaccaggag cttctggaca tcaagctggc cctggacatg gagatccacg |
| 1321 | cctaccgcaa gctcttggag ggcgaggagg agaggctacg cctgtccccc agccctacct |
| 1381 | cgcagcgcag ccgtggccgt gcttcctctc actcatccca gacacagggt gggggcagcg |
| 1441 | tcaccaaaaa gcgcaaactg gagtccactg agagccgcag cagcttctca cagcacgcac |
| 1501 | gcactagcgg gcgcgtggcc gtggaggagg tggatgagga gggcaagttt gtccggctgc |
| 1561 | gcaacaagtc caatgaggac cagtccatgg gcaattggca gatcaagcgc cagaatggag |
| 1621 | atgatccctt gctgacttac cggttcccac caaagttcac cctgaaggct gggcaggtgg |
| 1681 | tgacgatctg ggctgcagga gctggggcca cccacagccc ccctaccgac ctggtgtgga |
| 1741 | aggcacagaa cacctggggc tgcgggaaca gcctgcgtac ggctctcatc aactccactg |
| 1801 | gggaagaagt ggccatgcgc aagctggtgc gctcagtgac tgtggttgag gacgacgagg |
| 1861 | atgaggatgg agatgacctg ctccatcacc accacggctc ccactgcagc agctcggggg |
| 1921 | accccgctga gtacaacctg cgctcgcgca ccgtgctgtg cgggacctgc gggcagcctg |
| 1981 | ccgacaaggc atctgccagc ggctcaggag cccagagccc ccagaactgc agcatcatgt |
| 2041 | aatctgggac ctgccaggca ggggtggggg tggaggcttc ctgcgtcctc ctcacctcat |
| 2101 | gcccaccccc tgccctgcac gtcatgggag ggggcttgaa gccaaagaaa aataaccctt |
| 2161 | tggttttttt cttctgtatt tttttttcta agagaagtta ttttctacag tggttttata |
| | ctgaaggaaa aacacaagca aaaaaaaaaa aaaaaaa |

What is claimed:

1. An accelerated Alzheimer's Disease (AD) model system comprising cultured neuronal progenitor cells, or iPSCs, that have been (i) engineered to express progerin and (ii) and subjected to cell culture conditions leading to neuronal differentiation and wherein said cultured cells develop one or more AD associated phenotypes.

2. The accelerated AD model system of claim 1 wherein the one or more AD associated phenotypes is observed in less than 6 weeks.

3. The accelerated AD model system of claim 2, wherein the one or more AD associated phenotypes is observed in 3-4 weeks.

4. The accelerated AD model system of claim 1, wherein the neuronal progenitor cells, or iPSCs, are cultured in 2D/3D culture conditions.

5. The accelerated AD model system of claim 1, wherein the neuronal progenitor cells, or iPSCs, further express a familial AD gene mutation.

6. The model system of claim 1, wherein the neuronal progenitor cells are ReNcell VM immortalized human neural progenitor cells.

7. The accelerated AD model system of claim 1, wherein the one or more AD associated phenotypes is selected from the group consisting of Tau phosphorylation, amyloid plaque accumulation, elevated levels of Aβ42 to Aβ40 ratio, increased cell death and cell cycle re-entry.

8. A method for deriving an accelerated AD model system comprising (i) culturing progerin expressing neuronal progenitor cells or iPSCs optionally expressing progerin; (ii) and subjecting said cells to culture conditions leading to neuronal differentiation wherein said cultured cells develop one or more AD associated phenotypes.

9. The method of claim 8, wherein the one or more AD associated phenotypes is observed in less than 6 weeks.

10. The method of claim 9, wherein the one or more AD associated phenotypes is observed in 3-4 weeks.

11. The method of claim 8, wherein the neuronal progenitor cells, or iPSCs, are cultured in 2D/3D culture conditions.

12. The method of claim 8, wherein the neuronal progenitor cells, or iPSCs, further express a familial AD gene mutation.

13. The method of claim 8, wherein the neuronal progenitor cells are ReNcell VM immortalized human neural progenitor cells.

14. The method of claim 8, wherein the one or more cultured AD associated phenotypes is selected from the group consisting of Tau phosphorylation, amyloid plaque accumulation, elevated levels of Aβ42 to Aβ40 ration, increased cell death and cell cycle re-entry.

15. The method of claim 8, for use in the diagnosing or prognosing of AD in a subject wherein the iPSCs are derived from said subject and wherein detection of an increase in the level of AD associated phenotypes relative to a control biological sample indicates that the subject has AD, or is at risk of developing AD.

16. The method of claim 8, for use in identifying a therapeutic agent useful for treating AD wherein said therapeutic agent corrects an AD associated phenotype comprising the additional step of contacting said cultured neuronal progenitor cells, or iPSCs, with a test therapeutic agent and identifying a therapeutic agent as correcting the AD associated phenotype if a decrease in the level of AD associated phenotypes is detected relative to a control sample.

17. The method of claim 8, for use in the production of culture media containing aging factors wherein said method further comprises the step of harvesting of said culture media.

18. Culture media obtained using the method of claim 17.

19. A kit for diagnosing or prognosing AD in a subject, identifying a subject at risk of development of AD, or prescribing a therapeutic regimen or predicting benefit from therapy in a subject having AD, the kit comprising one or more agents which comprises one or more containers for collecting and/or holding a biological sample, wherein said biological sample comprises cultured neuronal progenitor cells, or iPSCs, that have been (i) engineered to express progerin and (ii) and subjected to cell culture conditions leading to neuronal differentiation, as well as reagents for detecting one or more AD associated phenotypes and an instruction for its use.

20. The kit of claim 19, wherein the one or more AD associated phenotypes is selected from the group consisting of Tau phosphorylation, amyloid plaque accumulation, elevated levels of Aβ42 to Aβ40 ration, increased cell death and cell cycle re-entry.


SEQUENCE LISTING (24 sequences)

SEQ ID NO: 1 - protein, 614 aa, Homo sapiens
  SITE 1..614: note = Progerin
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHGSHCSSSGDPAEYNLRSRTVLCGTCGQPADKASASGSGAQSPQNCSIM

SEQ ID NO: 2 - DNA, 2257 bp, Homo sapiens
  misc_feature 1..2257: note = nucleic acid encoding the progerin protein of SEQ ID NO:1
  source 1..2257: mol_type = genomic DNA
gagcgccgcacctacaccagccaacccagatcccgaggtccgacagcgcccggcccagatccccacgcctgccaggagcaagccgagagccagccggccggcgcactccgactccgagcagtctctgtccttcgacccgagccccgcgccctttccgggacccctgccccgcgggcagcgctgccaacctgccggccatggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaagcttgaggcagccctaggtgaggccaagaagcaacttcaggatgagatgctgcggcgggtggatgctgagaacaggctgcagaccatgaaggaggaactggacttccagaagaacatctacagtgaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaagctggacaatgccaggcagtctgctgagaggaacagcaacctggtgggggctgcccacgaggagctgcagcagtcgcgcatccgcatcgacagcctctctgcccagctcagccagctccagaagcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggaccagtccatgggcaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacggctcccactgcagcagctcgggggaccccgctgagtacaacctgcgctcgcgcaccgtgctgtgcgggacctgcgggcagcctgccgacaaggcatctgccagcggctcaggagcccagagcccccagaactgcagcatcatgtaatctgggacctgccaggcaggggtgggggtggaggcttcctgcgtcctcctcacctcatgcccaccccctgccctgcacgtcatgggagggggcttgaagccaaagaaaaataaccctttggtttttttcttctgtatttttttttctaagagaagttattttctacagtggttttatactgaaggaaaaacacaagcaaaaaaaaaaaaaaaaaa

SEQ ID NO: 3 - DNA, 20 bp, synthetic construct
  misc_feature 1..20: note = Synthetic: ACTB
  source 1..20: mol_type = other DNA
ctggaacggtgaaggtgaca

SEQ ID NO: 4 - DNA, 23 bp, synthetic construct
  misc_feature 1..23: note = Synthetic: ACTB
  source 1..23: mol_type = other DNA
aagggacttcctgtaacaatgca

SEQ ID NO: 5 - DNA, 22 bp, synthetic construct
  misc_feature 1..22: note = Synthetic: Lamin A
  source 1..22: mol_type = other DNA
gcaacaagtccaatgaggacca

SEQ ID NO: 6 - DNA, 30 bp, synthetic construct
  misc_feature 1..30: note = Synthetic: Lamin A
  source 1..30: mol_type = other DNA
catgatgctgcagttctgggggctctggat

SEQ ID NO: 7 - DNA, 21 bp, synthetic construct
  misc_feature 1..21: note = Synthetic: Lamin C
  source 1..21: mol_type = other DNA
ctcagtgactgtggttgagga

SEQ ID NO: 8 - DNA, 17 bp, synthetic construct
  misc_feature 1..17: note = Synthetic: Lamin C
  source 1..17: mol_type = other DNA
agtgcaggctcggcctc

SEQ ID NO: 9 - DNA, 22 bp, synthetic construct
  misc_feature 1..22: note = Synthetic: Lamin B1
  source 1..22: mol_type = other DNA
ggagaatcgttgtcagagcctt

SEQ ID NO: 10 - DNA, 20 bp, synthetic construct
  misc_feature 1..20: note = Synthetic: Lamin B1
  source 1..20: mol_type = other DNA
tgcggctttccatcagttct

SEQ ID NO: 11 - DNA, 20 bp, synthetic construct
  misc_feature 1..20: note = Synthetic: APP
  source 1..20: mol_type = other DNA
tgggttcaaacaaaggtgca

SEQ ID NO: 12 - DNA, 20 bp, synthetic construct
  misc_feature 1..20: note = Synthetic: APP
  source 1..20: mol_type = other DNA
gttctgctgcatcttggaca

SEQ ID NO: 13 - DNA, 22 bp, synthetic construct
  misc_feature 1..22: note = Synthetic: PSEN1
  source 1..22: mol_type = other DNA
gcagtatcctcgctggtgaaga

SEQ ID NO: 14 - DNA, 23 bp, synthetic construct
  misc_feature 1..23: note = Synthetic: PSEN1
  source 1..23: mol_type = other DNA
caggctatggttgtgttccagtc

SEQ ID NO: 15 - DNA, 18 bp, synthetic construct
  misc_feature 1..18: note = Synthetic: P16
  source 1..18: mol_type = other DNA
gagcagcatggagccttc

SEQ ID NO: 16 - DNA, 20 bp, synthetic construct
  misc_feature 1..20: note = Synthetic: P16
  source 1..20: mol_type = other DNA
cgtaactattcggtgcgttg

SEQ ID NO: 17 - DNA, 22 bp, synthetic construct
  misc_feature 1..22: note = Synthetic: Cdk4
  source 1..22: mol_type = other DNA
ccatcagcacagttcgtgaggt

SEQ ID NO: 18 - DNA, 22 bp, synthetic construct
  misc_feature 1..22: note = Synthetic: Cdk4
  source 1..22: mol_type = other DNA
tcagttcgggatgtggcacaga

SEQ ID NO: 19 - DNA, 20 bp, synthetic construct
  misc_feature 1..20: note = Synthetic: Cdk6
  source 1..20: mol_type = other DNA
ccaggcaggcttttcattca

SEQ ID NO: 20 - DNA, 20 bp, synthetic construct
  misc_feature 1..20: note = Synthetic: Cdk6
  source 1..20: mol_type = other DNA
aagtatgggtgagacagggc

SEQ ID NO: 21 - DNA, 39 bp, synthetic construct
  misc_feature 1..39: note = Synthetic: Telomere
  source 1..39: mol_type = other DNA
cggtttgtttgggtttgggtttgggtttgggtttgggtt

SEQ ID NO: 22 - DNA, 39 bp, synthetic construct
  misc_feature 1..39: note = Synthetic: Telomere
  source 1..39: mol_type = other DNA
ggcttgccttacccttacccttacccttacccttaccct

SEQ ID NO: 23 - DNA, 27 bp, synthetic construct
  misc_feature 1..27: note = Synthetic: Beta-globin
  source 1..27: mol_type = other DNA
gcttctgacacaactgtgttcactagc

SEQ ID NO: 24 - DNA, 23 bp, synthetic construct
  misc_feature 1..23: note = Synthetic: Beta-globin
  source 1..23: mol_type = other DNA
caccaacttcatccacgttcacc